(19) United States
(12) Patent Application Publication
(10) Pub. No.: US 2013/0202549 A1
(43) Pub. Date: Aug. 8, 2013

(54) USE OF DPAS AND CO-POLYMERS FOR HYPERLIPIDEMIA AND ATHEROSCLEROSIS ALONG WITH REDUCING FEEDING RATE AND ADIPOSE TISSUE WEIGHT OF OBESITY ANIMAL

(71) Applicant: Ing-Jun Chen, Kaohsiung (TW)

(72) Inventor: Ing-Jun Chen, Kaohsiung (TW)

(21) Appl. No.: 13/605,889

(22) Filed: Sep. 6, 2012

(30) Foreign Application Priority Data
Sep. 6, 2011 (TW) 100132154

(51) Int. Cl. C07D 473/08; A61K 47/48

(52) U.S. Cl. CPC C07D 473/08; A61K 47/4823; A61K 47/48176; A61K 47/482
USPC 424/78.29; 514/252.16; 514/54; 424/78.17; 514/59; 514/56; 544/267; 536/31; 536/51; 536/21; 525/281; 525/450; 525/436

(57) ABSTRACT

The disubstituted piperazine analogs (DPAs) derivative compound and DPAs amine complex compound disclosed in the present aspects have characterized by presented pharmaceutics having functions to improve lipolysis, such as inhibiting obesity hyperlipidemia, and atherosclerosis.

### CROSS REFERENCE TO RELATED APPLICATIONS

This application claims benefit under 35 U.S.C. §119 of Taiwanese Patent Application No. 100132154, filed on Sep. 6, 2011, the contents of which are incorporated by reference as if fully set forth herein in its entirety.

### FIELD OF THE INVENTION

The present aspects relates to DPAs derivative compound capable of inhibiting obesity, and more particularly to those DPAs derivative compound capable of inhibiting feeding-rate of obesity, hyperlipidemia, atherosclerosis, over weight of adipose tissues and maintaining metabolic homeostasis.

### BACKGROUND OF THE INVENTION

The present aspects provides synthesized by the KMUPs amine complex compound and a carboxylic acid derivative of one selected from a group consisting of a statin, a non-steroid anti-inflammatory (NSAIDs) and an anti-asthmatic drug. The pharmaceutical compositions for a treatment of an interstitial lung disease have applied as Ser. No. 11/857,483 filed on Sep. 19, 2007.

### SUMMARY OF THE INVENTION

In accordance with an aspect of the present aspects, the method for inhibiting obesity, comprising a step of:

administering to a subject in need an effective amount of a pharmaceutical composition comprising one of a DPAs derivative compound and a DPAs amine complex compound.

In accordance with another aspect of the present aspects, the method for reducing feeding-rate of obesity and weight of adipose tissues, comprising a step of:

administering to a subject in need an effective amount of a pharmaceutical composition comprising one of a DPAs derivative compound and a DPAs amine complex compound.

In the disubstituted piperazine analogs (DPAs) derivative compound is represented by

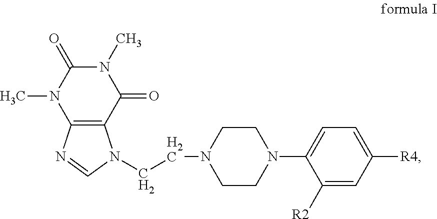

wherein R2 and R4 are each selected independently from the group consisting of a C1˜C5 alkoxy group, a hydrogen, a nitro group, and a halogen atom, and halogen atom is one selected from a group consisting of a fluorine, a chlorine, a bromine and an iodine.

The disubstituted piperazine analogs (DPAs) amine complex compound is represented by

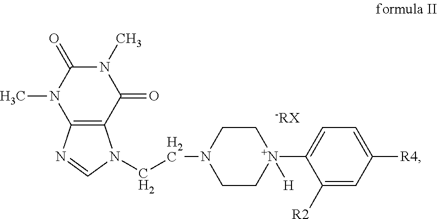

Wherein R2 and R4 are each selected independently from the group consisting of a C1˜C5 alkoxy group, a hydrogen, a nitro group, and a halogen atom;
RX contains a carboxylic group which donated from one of a Statin derivative, a co-polymer, a poly-γ-polyglutamic acid (γ-PGA) derivative, sodium carboxyl methylcellulose (sodium CMC) and a combination thereof; and
⁻RX substituent is an anion of the carboxylic group carrying a negative charge; and
halogen atom is one selected from a group consisting of a fluorine, a chlorine, a bromine and an iodine.

The above objects and advantages of the present aspects will become more readily apparent to those ordinarily skilled in the art after reviewing the following detailed descriptions and accompanying drawings, in which:

### BRIEF DESCRIPTION OF THE DRAWINGS

FIG. 1A-1D show the effects of mevalonate on HMGR expression in mice liver

FIG. 1A shows that effects of HMGR expression

FIG. 1B shows that effects of ROCK

FIG. 1C shows that effects of PPAR-γ expression

FIG. 1D shows that effects of ABCA1

FIG. 2A-2B show the effects of DPA-1 and simvastatin on HMGR expression in HepG2 cells.

FIG. 2A shows that effects of DPA-1

FIG. 2B shows that effects of simvastatin

FIG. 2C shows that effects of mevalonate

FIG. 2D shows that effect of DPA-1 and simvastatin

FIG. 3 shows the effects of DPA-1 on eNOS expression in HepG2 cells

FIG. 4A-4C show the effect of DPA-1 and simvastatin on ROCK expression in HepG2 cells

FIG. 4A shows that effects of membrane/cytosol

FIG. 4B shows that effects of DPA-1

FIG. 4C shows that effects of simvastatin

FIG. 5A-5C show the effects of DPA-1 and simvastatin on PPAR-γ expression in HepG2 cells

FIG. 5A shows that effects of DPA-1

FIG. 5B shows that effects of simvastatin

FIG. 5C shows that effects of DPA-1

FIG. 5D shows that effects of simvastatin

FIG. 6A-6B show the effects of DPA-1 and simvastatin on ABCA1 and ApoA1 expression in HepG2 cells

FIG. 6A shows that effects of DPA-1

FIG. 6B shows that effects of simvastatin

FIG. 7A-7C show the effects of isoprenoids alone or with DPA-1 on ROCK expression in HepG2 cells

FIG. 7A shows the effects of ROCK expression

FIG. 7B shows the effects of PPAR-γ expression

FIG. 7C shows the effects of ABCA1 expression

FIG. 8A-8C show the effects of isoprenoids alone or with DPA-1 on Rho A and ROCK II expression in HepG2 cells

FIG. 8A shows that effects of membrane/cytosol

FIG. 8B shows that effect of DPA-1 and GGPP

FIG. 8C shows the effect of DPA-1 and FPP

FIG. 9A-9E show the effects of isoprenoids alone or with DPA-1 on

PPARγ and ABCA1 expression in HepG2 cells

FIG. 9A shows that effect of DPA-1 and FPP on PPAR-γ expression

FIG. 9B shows that effects of DPA-1 and FPP on ABCA1 expression

FIG. 9C shows the effect of DPA-1 and GGPP on PPAR-γ expression

FIG. 9D shows the effect of DPA-1 and GGPP on ABCA1 expression

FIG. 9E shows the effect of simvastatin and GGPP on PPAR-γ expression

FIG. 10 show the effects of isoprenoids alone or with DPA-1 on LXR-α expression in HepG2 cells

### DETAILED DESCRIPTION OF THE PREFERRED EMBODIMENT

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

The benefits of statins or DPA-1 in cardiovascular system may not only be due to their cholesterol-lowering effects, but also, to their pleiotropic effects, predominantly due to inhibiting isoprenoid synthesis, the products of which are important lipid attachments for intracellular signaling (Murtola et al., 2008). Besides cholesterol, application of mevalonate to liver cells results in biosynthesis of the isoprenoid compounds farnesyl pyrophosphate (FPP) and geranylgeranyl pyrophosphate (GGPP), levels of which are reduced by statins. In addition, application of GGPP, FPP and mevanolate to cells can stop the functionally processing of HMGR; increased levels of GTP-RhoA after GGPP depletion have enhanced Rho effector signaling (Waiczies et al., 2007).

ROCK has newly emerged as the principal mechanisms underlying the pleiotropic effects of statins and DPA-1 has been described to inhibit RhoA/ROCK via NO/cGMP-dependent pathway. eNOS, the main source of endothelium-derived NO, related to RhoA/ROCK, appears to be a therapeutic target in lipid-lowering and treatment of atherosclerosis. Decrease in Rho GTPase responses increases the production and bioavailability of endothelium-derived NO. The regulation of eNOS by Rho GTPases is an important mechanism underlying the cardiovascular protective effect. Increase of eNOS/cGMP and decrease of RhoA/ROCK contribute to pleiotropic activities of HMGR antagonists.

Anti-hypercholesterolemia agent is required to increase HDL, because low HDL cholesterol levels constitute an independent risk factor to cardiovascular disease; increased HDL can protects against atherosclerosis through the action reversing cholesterol transport pathway (Brewer, 2004). Liver X receptor (LXRα ) regulates the expression of ABCA1, which is involved in the HDL-related reverse cholesterol transport (RCT) pathway (Ando et al., 2004).

Peroxisome proliferator activated receptors (PPARs) are found in all mammalian species and each subclass has unique tissue specificity and functions (Peters et al., 2005). Among them, PPAR-γ is expressed in controls of adipocyte differentiation, fat storage and inflammation (Tontonoz et al., 1995). Cellular cholesterol metabolism is accomplished, in part, by PPARs and LXRα (Argmann et al., 2005). Activation of isoprenoid can produce FPP and GGPP that has been demonstrated to inhibit ABCA1, directly through antagonism of LXRα and indirectly through activated geranylgeranylation of RhoA (Argmann et al., 2005).

Disubstituted piperazine analogs (DPAs) derivative compound is represented by

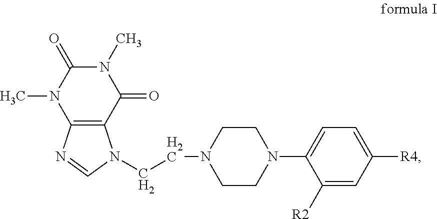

wherein R2 and R4 are each selected independently from the group consisting of a C1˜C5 alkoxy group, a hydrogen, a nitro group, and a halogen atom, and halogen atom is one selected from a group consisting of a fluorine, a chlorine, a bromine and an iodine.

Preferably, in one embodiment, the compound of formula I is DPA-1, wherein R₂ is chlorine atom and R₄ is hydrogen, which has the generally chemical name 7-[2-[4-(2-chlorophenyl)piperazinyl]ethyl]-1,3-dimethylxanthine. The compound of formula I is DPA-2, wherein R₂ is methoxy group and R₄ is hydrogen, which has the chemical name 7-[2-[4-(4-(2-methoxybenzene)piperazinyl]ethyl]-1,3-dimethylxanthine. In another embodiment, the compound of formula I also is DPA-3, wherein R₂ is hydrogen and R₄ is nitro group, which has the chemical name 7-[2-[4-(4-nitrobenzene)piperazinyl]ethyl]-1,3-dimethylxanthine. In another embodiment, the compound of formula I also is DPA-4, wherein R₂ is nitro group and R₄ is hydrogen, which has the chemical name 7-[2-[4-(2-nitrobenzene)piperazinyl]ethyl]-1,3 -dimethylxanthine

A DPAs amine complex compound having a formula II,

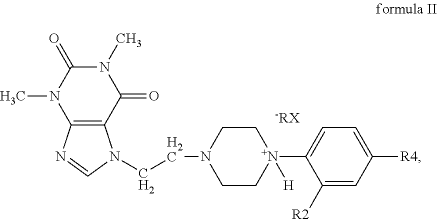

wherein R2 and R4 are each selected independently from the group consisting of a C1˜C5 alkoxy group, a hydrogen, a nitro group, and a halogen atom. The above-mentioned halogen refers to fluorine, chlorine, bromine and iodine. RX contains a carboxylic group which donated from a group consisting of a member of Statin derivative, poly-γ-polyglutamic acid (γ-PGA) derivative, co-polymer, sodium carboxyl methylcellulose (sodium CMC) and a combination thereof; ⁻RX can be an anion of the above-mentioned groups carrying a negative charge.

Preferably, statin derivative is one selected from a group consisting of Atorvastatin, Cerivastatin, Fluvastatin, Lovastatin, Mevastatin, Pravastatin, Rosuvastatin, Simvastatin (Sim.) and a combination thereof Co-polymer is one selected from a group consisting of hyaluronic acid (HA), polyacrylic acid (PAA), polymethacrylates (PMMA), Eudragit, dextran sulfate, heparan sulfate, polylactic acid or polylactide (PLA), polylactic acid sodium (PLA sodium), polyglycolic acid sodium (pga sodium) and a combination thereof Poly-γ-polyglutamic acid (γ-PGA) derivative is one selected from a group consisting of alginate sodium, poly-γ-polyglutamic acid sodium (γ-PGA sodium), poly-γ-polyglutamic acid (γ-PGA), alginate-poly-lysine-alginate (APA) and a combination thereof Hyaluronic Acid (HA) is a polymer, which are composed of alternating units of N-acetyl glucosamine complex compound (NAG), D-glucuronic acid and a combination thereof Eudragit is a trade name of series co-polymer derived from esters of acrylic and methacrylate acid.

Both DPAs derivative compound and DPAs amine complex compound as used herein refers to one selected from a group consisting of a theophylline-based moiety compound and its pharmaceutical acceptable salts. Preferably, the pharmaceutical composition further includes at least one of a pharmaceutically acceptable carrier and an excipient. Preferably, a theophylline-based moiety compound derivative, which is obtained by reacting theophylline compound with piperazine compound and is then recrystallized the intermediate therefrom, is provided in the present aspects.

Preferably, in one embodiment, the compound of formula II is DPA-1-RX, wherein R₂ is chlorine atom and R₄ is hydrogen atom, which has the generally chemical name 7-[2-[4-(2-chlorophenyl) piperazinyl]ethyl]-1,3-dimethylxanthine-RX. The compound of formula II is DPA-2-RX, wherein R₂ is methoxy group and R₄ is hydrogen, which has the chemical name 7-[2-[4-(2-methoxybenzene)piperazinyl]ethyl]-1,3-dimethylxanthine-RX. In another embodiment, the compound of formula II also is DPA-3-RX, wherein R₂ is hydrogen and R₄ is nitro group, which has the chemical name 7-[2-[4-(4-nitrobenzene)piperazinyl]ethyl]-1,3-dimethylxanthine-RX. In another embodiment, the compound of formula II also is DPA-4-RX, wherein R₂ is nitro group and R₄ is hydrogen, which has the chemical name 7-[2-[4-(2-nitrobenzene)piperazinyl]ethyl]-1,3-dimethylxanthine-RX.

To achieve the above purpose, formula II amine complex can be synthetically produced from the 2-chloroethyltheophylline compound and piperazine substituted compound.

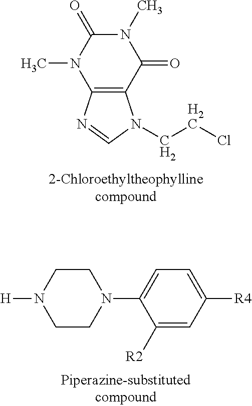

The compounds of formula II amine complex set forth in the examples below were prepared using the following general procedures as indicated.

The general procedure 1 includes steps of dissolving 2-chloroethyl theophylline and piperazine substituted compound in hydrous ethanol solution, and the amount of reagent should be conjugated depending on the molecular weight percentage. After adding the strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain DPA-1 HCl with a white crystal.

The general procedure 2 includes steps of dissolving 2-Chloroethyl theophylline and piperazine-substituted compounds in hydrous ethanol solution, and the amount of reagent should be conjugated depending on the molecular weight percentage. Then, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl ), kept at 50° C. to 60° C. to make a saturated solution (pH 1.2). The saturated solution was sequentially treated, decolorized with activated charcoal, filtered, deposited overnight and filtered to obtain DPA-1 HCl with a white crystal.

According to the general procedure 1 or 2, DPAs amine complex compound of formula II can be synthetically produced directly, from the 2-chloroethyltheophylline compound, piperazine substituted compound and carboxylic acid selected from the group of RX. Thereby, DPAs may represent DPAs derivative compound or DPAs amine complex compound, unless it explains specially. Preferably, in one embodiment, DPA-1 is dissolved in a mixture of ethanol and γ-Polyglutamic acid. The solution is reacted at warmer temperature, the methanol is added thereinto under room temperature, and the solution is incubated over night for crystallization and filtrated to obtain DPA-1-γ-Polyglutamic acid.

According to the above-mentioned aspect of the present aspects, DPAs amine complex compound are preferred to inhibit obesity. Specifically speaking, DPAs amine complex compound in one embodiment, as DPA-1-Atorvastatin, DPA-2-Atorvastatin, DPA-3-Atorvastatin, DPA-4-Atorvastatin, DPA-1-Cerivastatin, DPA-2-Cerivastatin, DPA-3-Cerivastatin, DPA-4-Cerivastatin, DPA-1-Fluvastatin, DPA-2-Fluvastatin, DPA-3-Fluvastatin, DPA-4-Fluvastatin, DPA-1-Lovastatin, DPA-2-Lovastatin, DPA-3-Lovastatin, DPA-4-Lovastatin, DPA-1-Mevastatin, DPA-2-Mevastatin, DPA-3-Mevastatin, DPA-4-Mevastatin, DPA-1-Pravastatin, DPA-2-Pravastatin, DPA-3-Pravastatin, DPA-4-Pravastatin, DPA-1-Rosuvastatin, DPA-2-Rosuvastatin, DPA-3 -Rosuvastatin, DPA-4-Rosuvastatin, DPA-1-Simvastatin, DPA-2-Simvastatin, DPA-3-Simvastatin, DPA-4-Simvastatin, DPA-1-CMC, DPA-2-CMC, DPA-3-CMC, DPA-4-CMC, DPA-1-hyaluronic acid, DPA-2-hyaluronic acid, DPA-3-hyaluronic acid, DPA-4-hyaluronic acid, DPA-1-polyacrylic acid, DPA-2-polyacrylic acid, DPA-3-polyacrylic acid, DPA-4-polyacrylic acid, DPA-1-Eudragit, DPA-2-Eudragit, DPA-3-Eudragit, DPA-4-Eudragit, DPA-1-polylactide, DPA-2-polylactide, DPA-3-polylactide, DPA-4-polylactide, DPA-1-polyglycolic acid, DPA-2-polyglycolic acid, DPA-3-polyglycolic acid, DPA-4-polyglycolic acid, DPA-1-dextran sulfate, DPA-2-dextran sulfate, DPA-3-dextran sulfate, DPA-4-dextran sulfate, DPA-1-heparan sulfate, DPA-2-heparan sulfate, DPA-3-heparan sulfate, DPA-4-heparan sulfate, DPA-1-alginate, DPA-2-alginate, DPA-3-alginate, DPA-4-alginate, DPA-1-γ-PGA, DPA-2-γ-PGA, DPA-3-γ-PGA, DPA-4-γ-PGA, DPA-1-APA, DPA-2-APA, DPA-3-APA, DPA-4-APA etc.

In accordance with a further aspect of the present aspects, depending on the desired clinical use and the effect, the adaptable administration method of DPAs pharmaceutical composition includes one selected from a group consisting of an oral administration, an intravenous injection, a subcutaneous injection, an intraperitoneal injection, an intramuscular injection and a sublingual administration. The DPAs pharmaceutical composition providing a medical effect for inhibiting obesity related condition or disease, and more particularly to inhibiting feeding-rate of obesity, hyperlipidemia, over weight of adipose tissues and maintaining metabolic homeostasis. In another embodiment, said obesity related condition means incorporate decrease adipose tissue and increase lean body mass, beneficial to consumers who are willing to eat lack fat of animal muscle food. The farmers also economically feed the animals by a less amount of foods in the presence of food-additives containing DPAs. Perferably, said, animal included human and non-human animal as meat poultry (beef, lamb, pork, chicken, dark, goose, pigeon, goat, and horse).

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself Each formulation prepared by combining the derivatives disclosed in the present aspects and the pharmaceutically acceptable carriers or excipients will not cause the undesired effect, allergy or other inappropriate effects while being administered to an animal or human. Accordingly, the derivatives disclosed in the present aspects in combination with the pharmaceutically acceptable carrier or excipients are adaptable in the clinical usage and in the human. A therapeutic effect can be achieved by using the dosage form in the present aspects by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylate methyl cellulose (CMC) or the analogous dispersing agents, such as methyl cellulose, ethyl cellulose and hydroxyl ethyl methyl cellulose (HEMC). Other carriers are common surfactant such as Tween and Spans or other analogous emulsion, or the pharmaceutically acceptable solid, liquid or other bio-avaliable enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier is generally used in the oral formulation. Taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present aspects may be formulated as suppositories for rectal or virginal administration.

The compound of the present aspects can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

The term “hyperlipemia or hyperlipidemia” used throughout the specification of the present invention refers to an imbalance between resting energy intake and energy expenditure. Such an increase in fat storage can occur independently of adipogenesis, i.e., the production of new fat cells. Furthermore, many complications of obesity and body weight gain result from improper storage of fat eg. total cholesterol, triglyceride, high density lipoprotein etc., in liver.

Obesity and fatty liver disease of animals is induced by free fatty acid secretion from visceral fat tissue through lipolysis of adipocytes. However, excess lipolysis of adipocytes, such as stimulated by β-agonist administration in obesity animal caused by high-fat-diet (HFD), hydrolyze intracellular triglyceride and accumulate fatty acid levels via hormone-sensitive lipase (HSL), enforcing fatty acids secretion toward extracellular domain and thus reducing the size of adipose tissues.

Adipose tissue, which is primarily composed of adipocytes, is crucial for maintaining energy and metabolic homeostasis. Obesity and fatty liver disease of animals is induced by free fatty acid secretion from visceral fat tissue through lipolysis of adipocytes. However, excess lipolysis of adipocytes, such as stimulated by β-agonist administration in obesity animal caused by high-fat-diet (HFD), hydrolyze intracellular triglyceride via hormone-sensitive lipase (HSL), enforcing fatty acids secretion toward extracellular domain and thus reducing the size or weight of adipose tissues. These events decrease adipose tissue and increase lean body mass, beneficial to consumers who are willing to eat lack fat of animal muscle food. The farmers also economically feed the animals by a less amount of foods in the presence of food-additives containing one of DPAs derivative compound and DPAs amine complex compound.

The components of adipose tissues majorly include epididymal fat pads was reduced by treatments with DPAs in HFD-induced obesity mice. This invention suggests that cGMP-enhancer DPAs inhibit the obesity, via restoring PPARγ -regulated eNOS and inactivating RhoA/ROCK to enhance the lipolysis of adipocytes and reduced adipose tissue weight.

Lipolysis Activity in 3-T-3 adipocytes and Protein Expression

Elevated cAMP/cGMP acts as second messengers to activate hormone sensitive lipase (HSL). HSL, the rate-limiting enzyme regulating adipocyte lipolysis, then catalyzes the hydrolysis of triglycerides and results in the release of glycerol and FFA (increased lipolysis). Phosphodiesterases (PDE) are enzymes that hydrolyze cAMP to 5′-AMP. This action results in a decrease in lipolysis. PDE inhibitors increase intracellular cAMP levels. 3-isobutyl-1-methylxanthine (IBMX), a non-specific inhibitor of cAMP phosphodiesterases (PDE), is used as referenced PDE inhibitors. Isoproterenol, a non-specific beta-adrenergic agonist is used as the positive control if your test compounds affect lipolysis via beta-adrenergic receptors. In this study, DPA-1, a xanthine analogue, was used as cGMP-enhancer to activate HSL. Increased expression of HSL and phosphorylated HSL (p-HSL) by DPA-1 indicates the increased lipolysis activity. In addition, PPAR/eNOS in adipocytes is up-regulated by DPA-1 and thus led to increased HSL and p-HSL.

Western blotting analyses on protein expression in 3-T-3 adipocytes Table 1 Protein expression of eNOS, PPAR and p-HSL

TABLE 1
Protein expression of adipocytes cultutred for 4 days

| Protein | PPAR-γ | eNOS | Rho-kinase | p-HSL |
|---|---|---|---|---|
| Control | 100% | 100% | 100% | 100% |
| DPA-1 (0.1 μM) | 123% | 121% | 82% | 120% |
| DPA-1 (1.0 μM) | 135% | 132% | 78% | 136% |
| DPA-1 (10 μM) | 142% | 138% | 75% | 144% |
| DPA-2 (10 μM) | 135% | 129% | 81% | 135% |
| DPA-3 (10 μM) | 140% | 131% | 79% | 138% |
| DPA-4 (10 μM) | 143% | 125% | 83% | 136% |

TABLE 2

| Without treatment | eNOS | PDE-5A | ROCKII |
|---|---|---|---|
| control (Vehicle) | 100 (%) | 100 (%) | 100 (%) |
| DPA-1 HCl | 155.16 ± 14.8 | 64.5 ± 4.5 | 42.5 ± 6.3 |
| DPA-1-CMC | 152.14 ± 6.3 | 58.7 ± 3.5 | 38.6 ± 2.7 |
| DPA-2 HCl | 147.21 ± 8.6 | 68.8 ± 5.3 | 40.8 ± 3.7 |
| DPA-3 HCl | 148.31 ± 7.5 | 63.6 ± 5.2 | 44.5 ± 2.9 |
| DPA-4 HCl | 135.24 ± 7.5 | 73.4 ± 3.4 | 37.6 ± 2.8 |
| DPA-1- | 131.50 ± 6.7 | 78.2 ± 3.6 | 56.6 ± 3.4 |
| Polyglutamic acid | | | |
| DPA-1-alginic acid | 155.16 ± 14.8 | 64.5 ± 4.5 | 42.5 ± 6.3 |

P < 0.05; significantly different from control (n = 5)

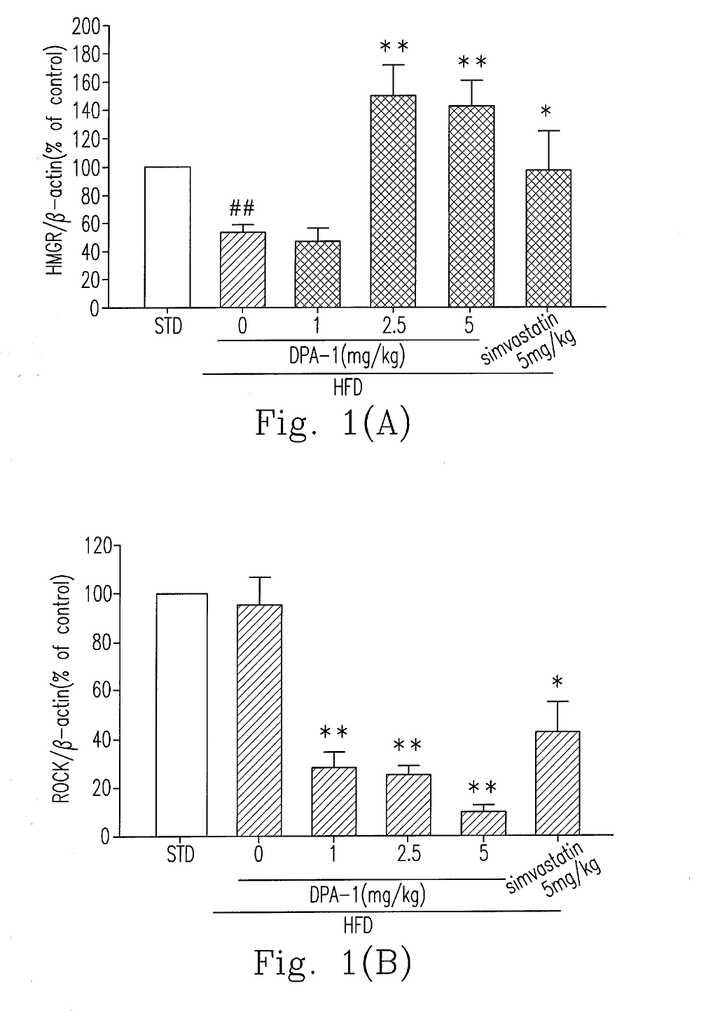

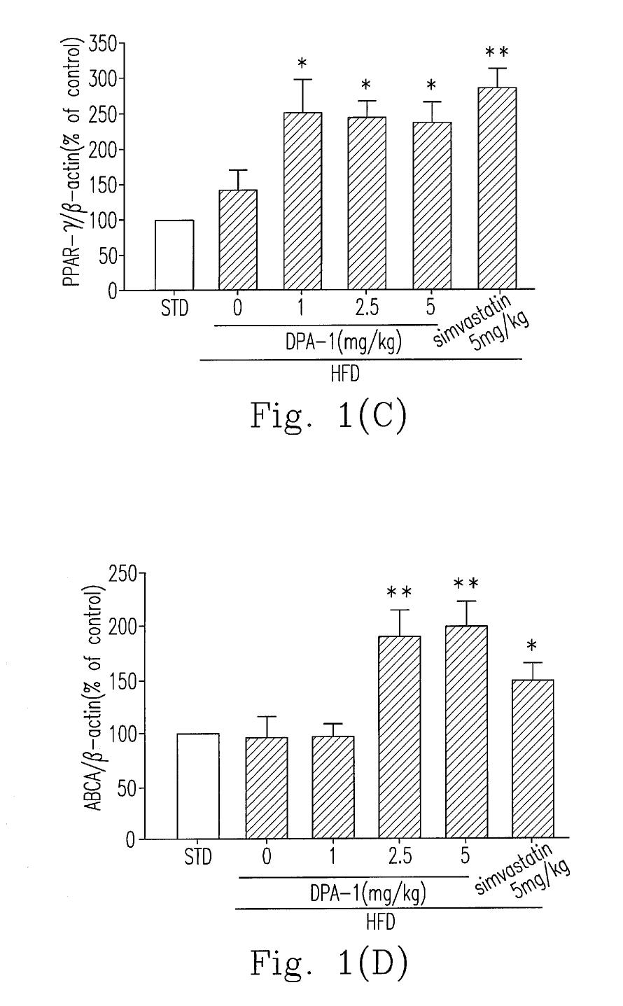

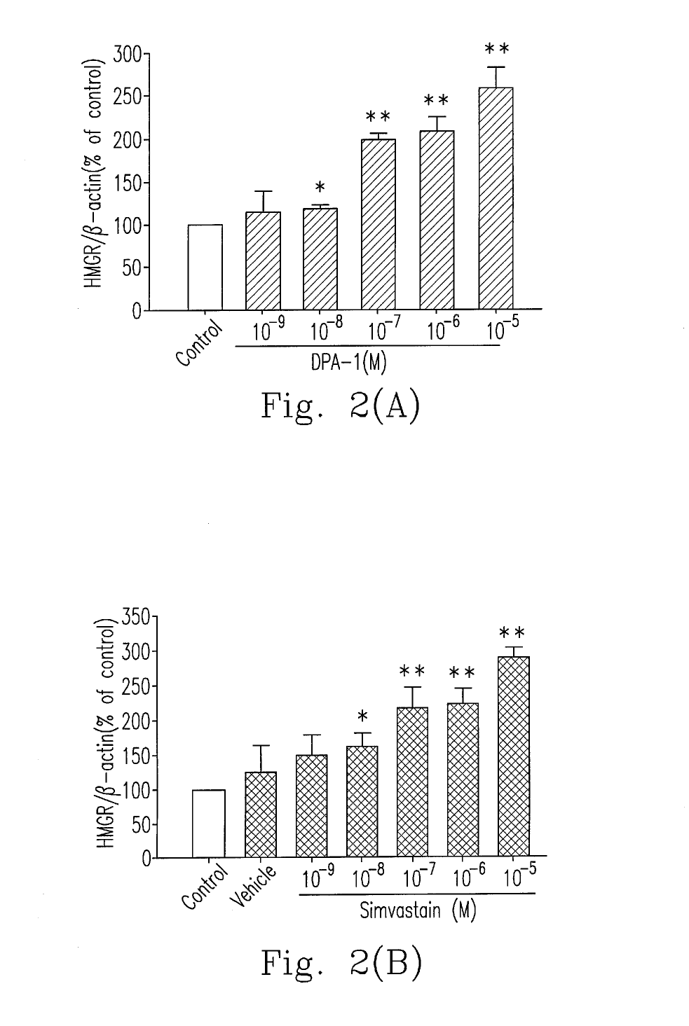

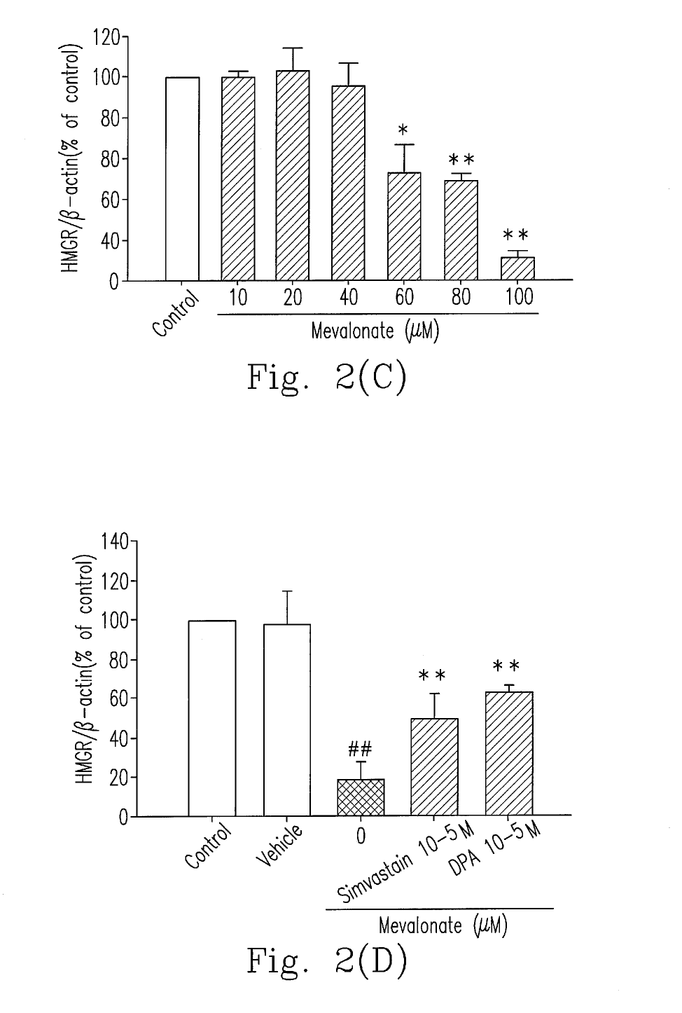

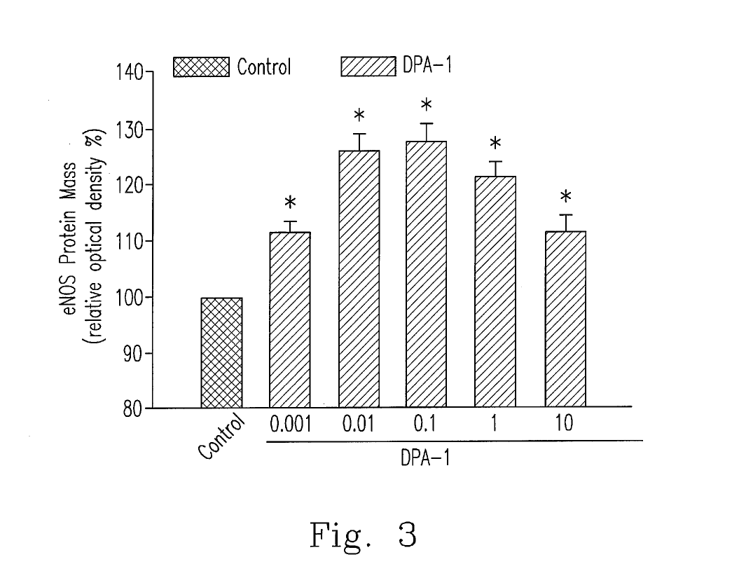

HepG2 cells were incubated with mevalonate (100 μM-1000 μM) for 24 h. HepG2 cells were preincubated for 1 h with mevalonate(100 μM) than treated DPA-1 (10⁻⁵ M) or simvastatin (10⁻⁵ M) to measure HMG-Co A Reductase protein expression by Western blotting analyses. FIG. 1 shows the effects of DPAs and simvastatin on HMG CoA Reductase (HMGR), ROCKII, PPARγ and ABCA1 expression of mice liver. HepG2 cells were incubated for 24 h with increasing doses of DPA-1 (10⁻⁹-10⁻⁵ M) (FIG. 2A) or simvastatin (10⁻⁹-10⁻⁵M) (FIG. 2B) by Western blot analysis. FIG. 2 shows the effects of DPAs and simvastatin and mevalonate on HMGR protein expression of HepG2 cell. HepG2 cells were incubated for 24 h with increasing doses of DPA-1 (10⁻⁹-10⁻⁵M) (FIG. 3A) by Western blot analysis. As shown in FIG. 3, eNOS is increased by DPA-1 (10⁻⁹˜10⁻⁴ μM) in HepG2 cells, contributing to NO-releasing and cGMP-enhancing activity.

Feed Back-Regulation on HMGR Expression

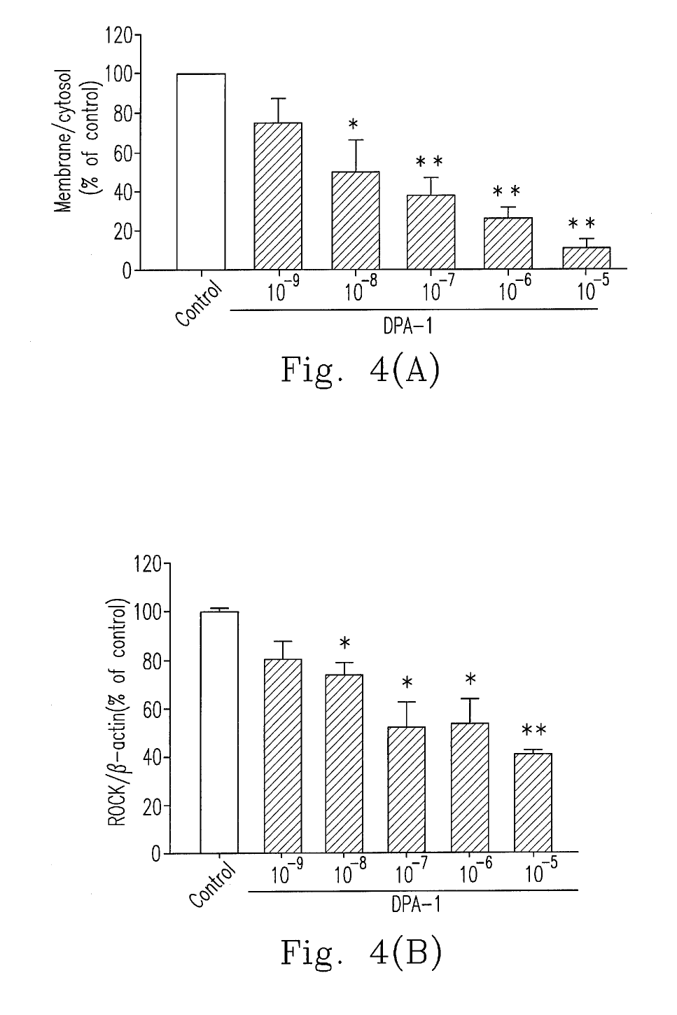

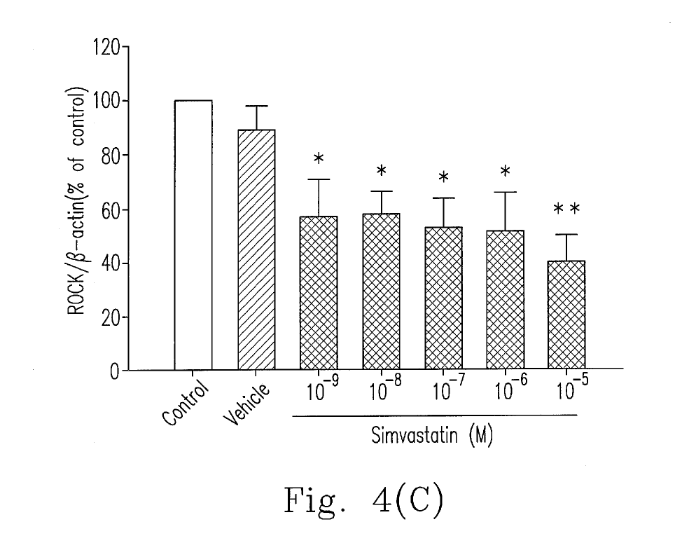

As shown in FIG. 4, application of mevalonate (10⁻⁵M) in HepG2 cells reduced expression of HMGR; its combination with DPA-1 (10⁻⁵M) reversed this inhibition, indicating their competitive feed-back regulation on HMGR.

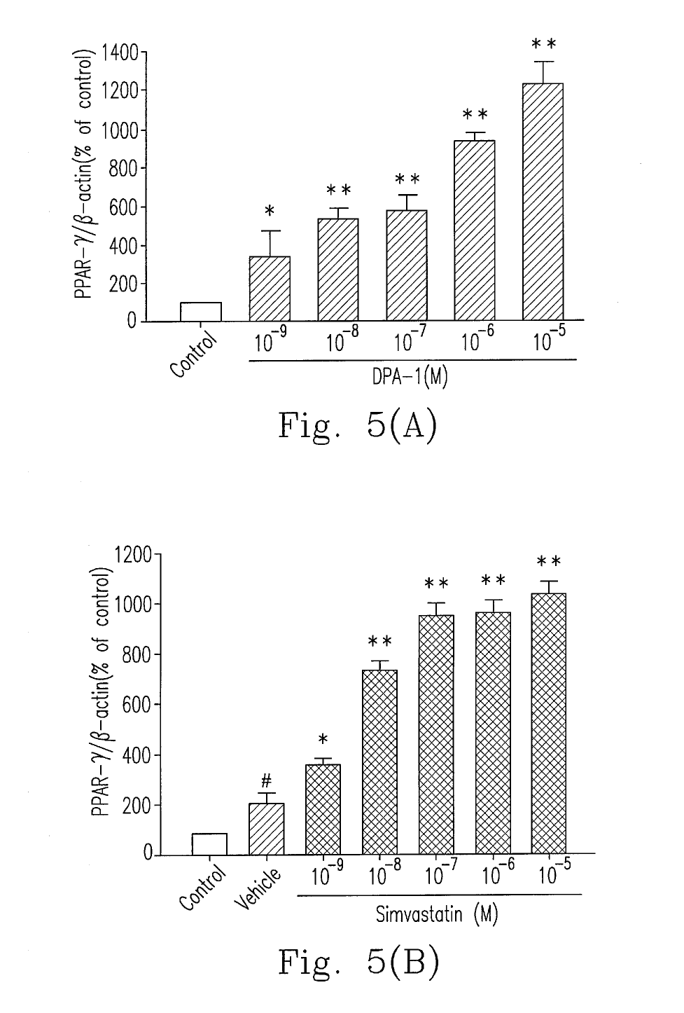

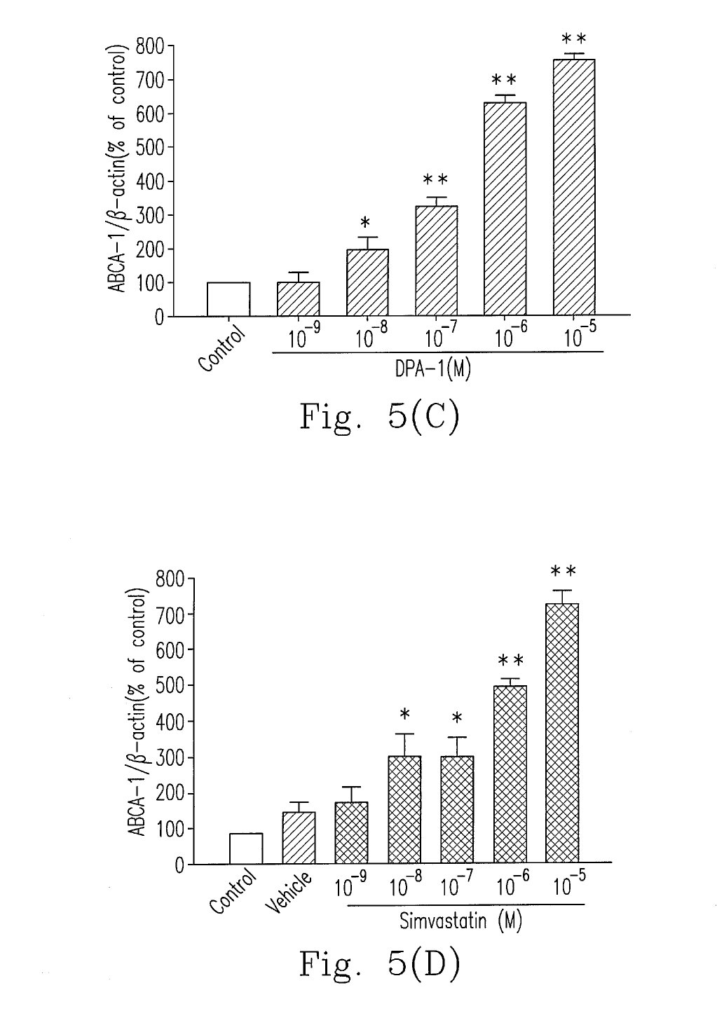

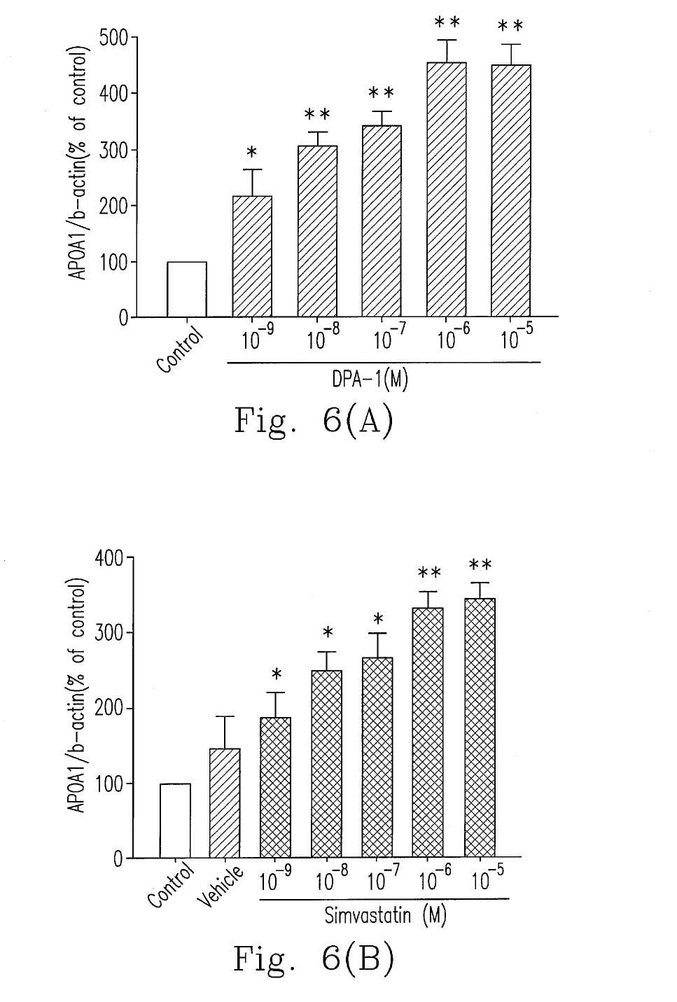

HepG2 cells were incubated with DPA-1 (10⁻⁹-10⁻⁵ M) (FIG. 4A) or simvastatin (10⁻⁹-10⁻⁵ M) (FIG. 4B) for 24 h. FIG. 4 shows the effects of DPA-1 and simvastatin on HMGR expressions in HepG2 cells. HepG2 cells were incubated with DPA-1 (10-9-10-5 M) (FIG. 5A) or simvastatin (10-9-10-5 M) (FIG. 5B) for 24 h. FIG. 5 shows the effects of DPA-1 and simvastatin on PPARγ and ABCA1 of HepG2 cells. HepG2 cells were incubated with DPA-1 (10⁻⁹-10⁻⁵ M) or simvastatin (10⁻⁹-10⁻⁵ M) for 24 h. FIG. 6 shows the effects of DPA-1 and simvastatin on ApoA 1.

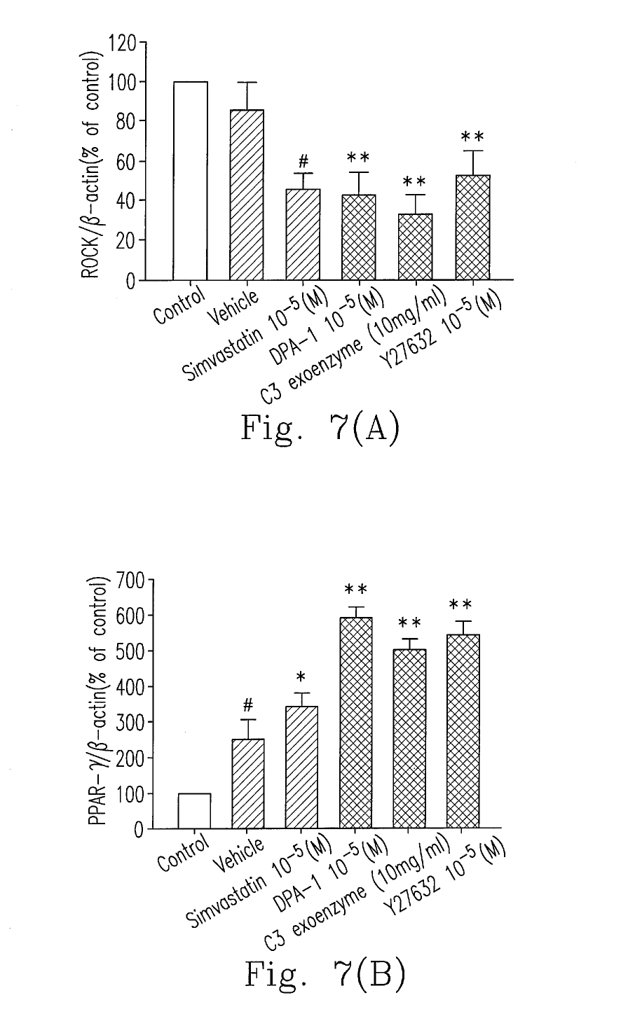

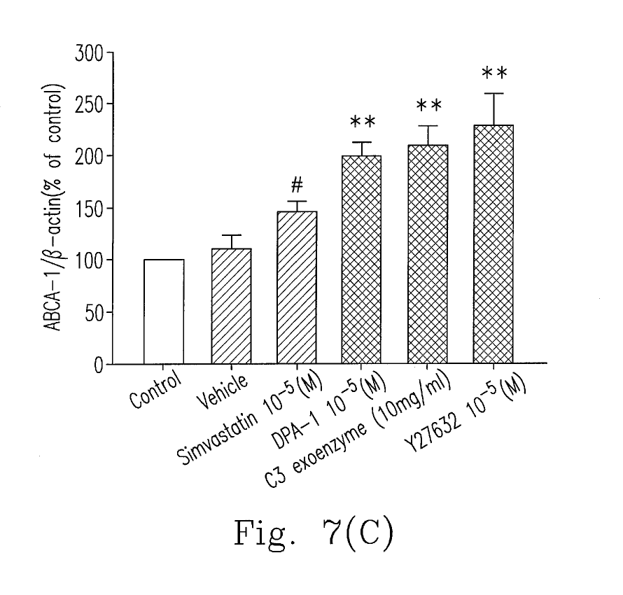

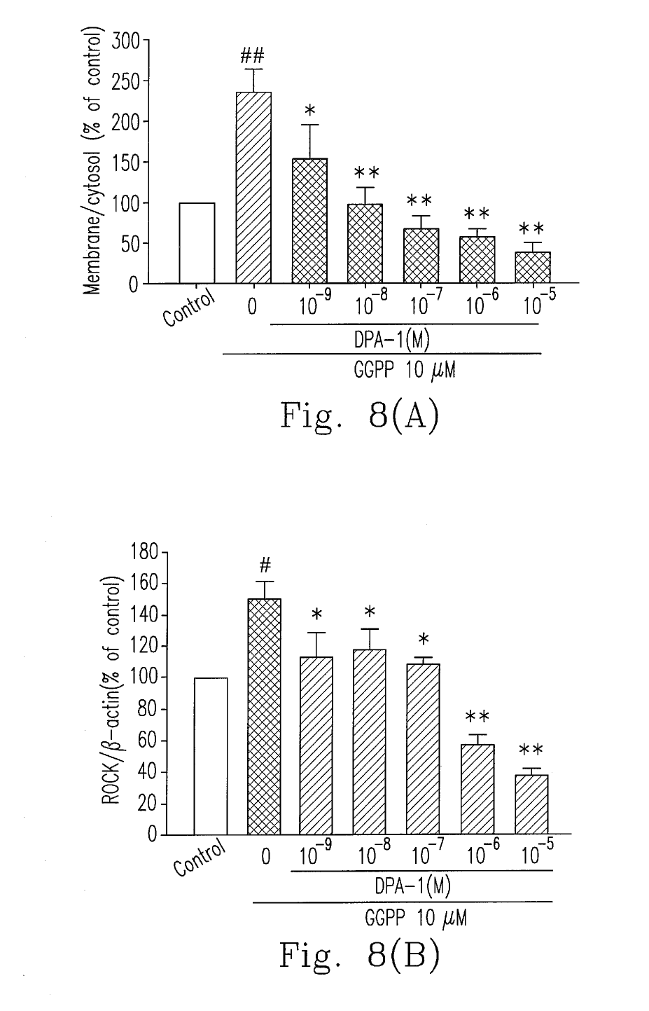

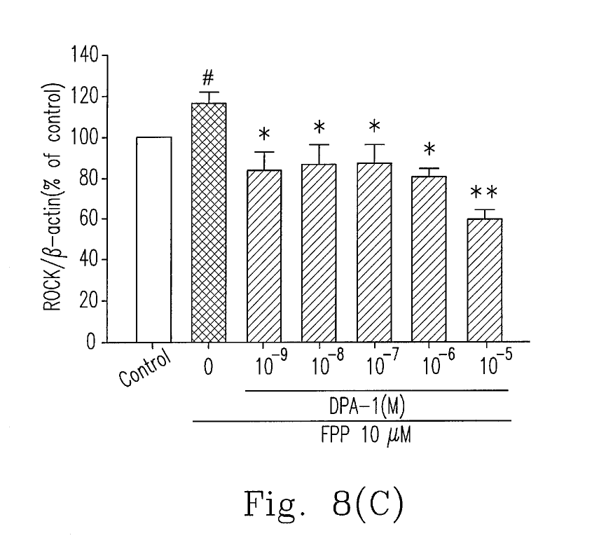

FIG. 7 shows the effects of DPA-1 and simvastati on ROCKII, PPAR and ABCA1. HepG2 cells were incubated with GGPP (10 μM) or FPP (10 μM) alone and GGPP (FIG. 7A) or FPP (FIG. 7B) with DPA-1 (10⁻⁹-10⁻⁵ M) for 24 h. HepG2 cells were incubated with GGPP (10 μM) or FPP (10 μM) alone and GGPP or FPP with DPA-1 (10⁻⁹-10⁻⁵M) for 24 h. FIG. 8 shows the effects of DPA-1 and simvastatin on Rho A and ROCK II.

RhoA/ROCKII Inactivation

In HepG2 cells, ROCKII (Rho kinase II) is the downstream effector of RhoA in hepatic cellular signaling. DPA-1 or simvastatin (10⁻⁹-10⁻⁵ M) concentration-dependently reduced ROCKII protein expression, indicating due to inhibited translocation of RhoA (FIG. 7).

Enhancement of ABCA1, ApoA1, LXRα and PPAR-γ

In HepG2 cells, DPA-1 or simvastatin (10⁻⁹-10⁻⁵M) increased cells levels of PPAR-γ and enhanced ABCA1 (ATP-binding cassette transporter A1) and ApoA1(apolipoprotein A-1) expression, indicating affecting the lipid metabolism toward HDL formation.

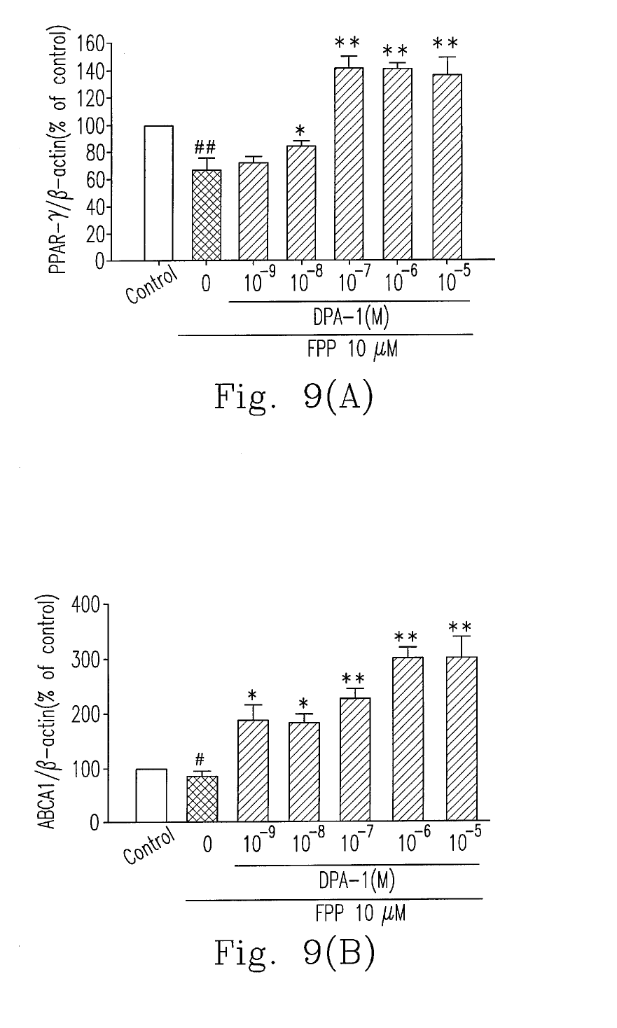

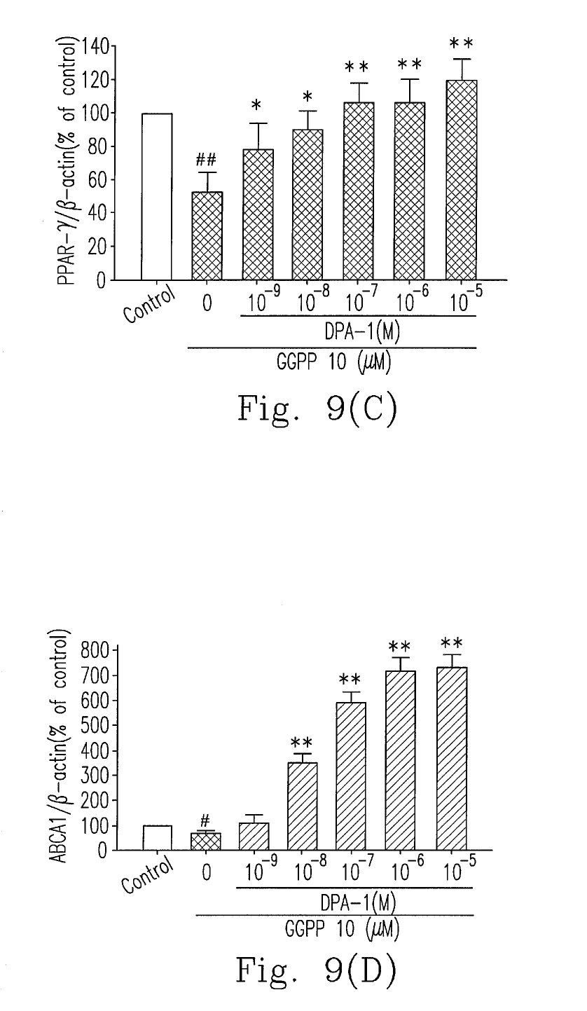

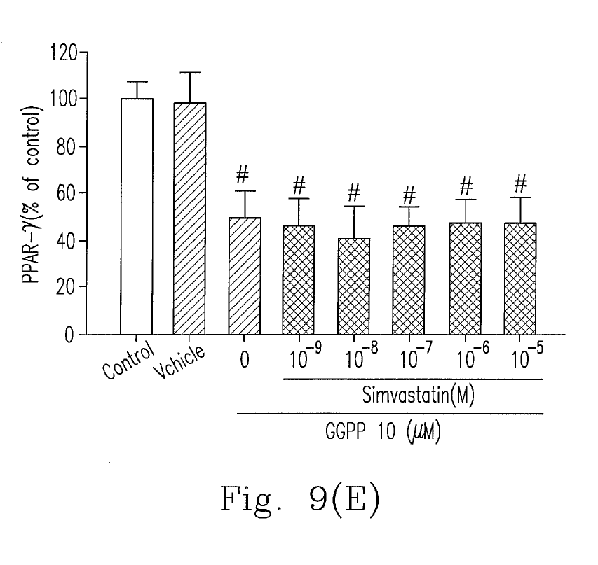

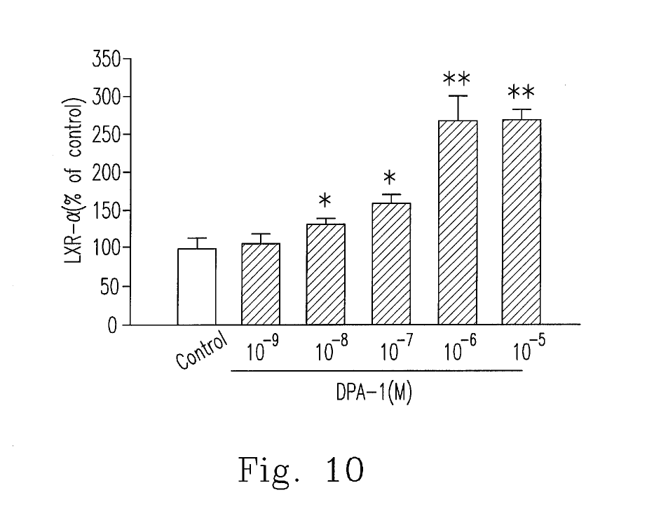

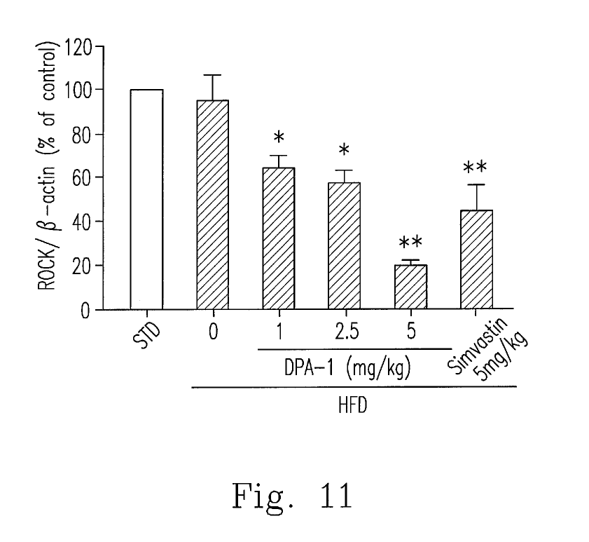

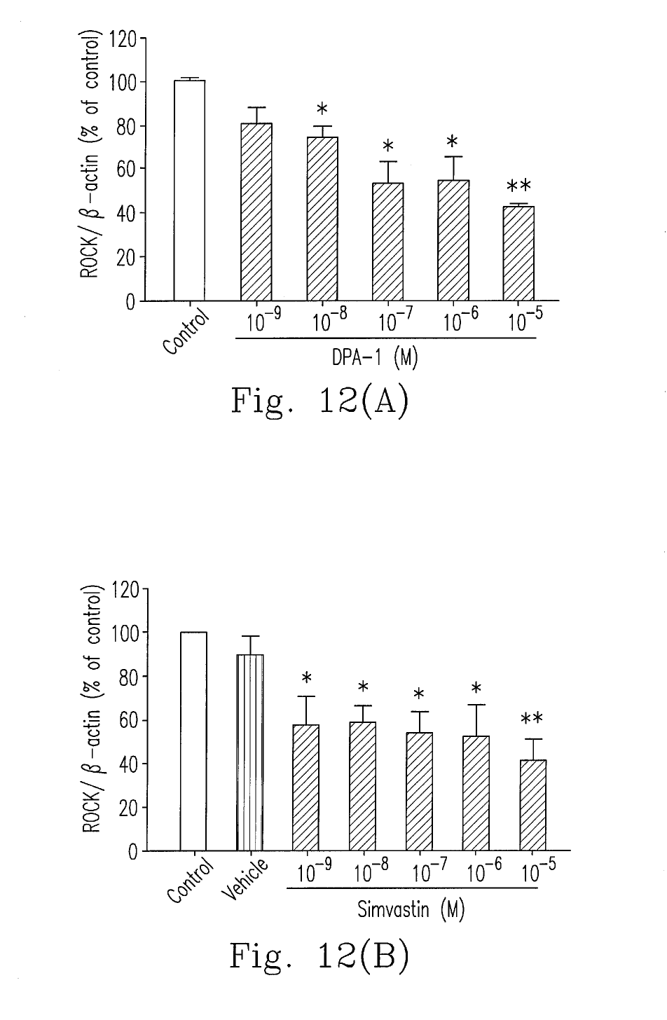

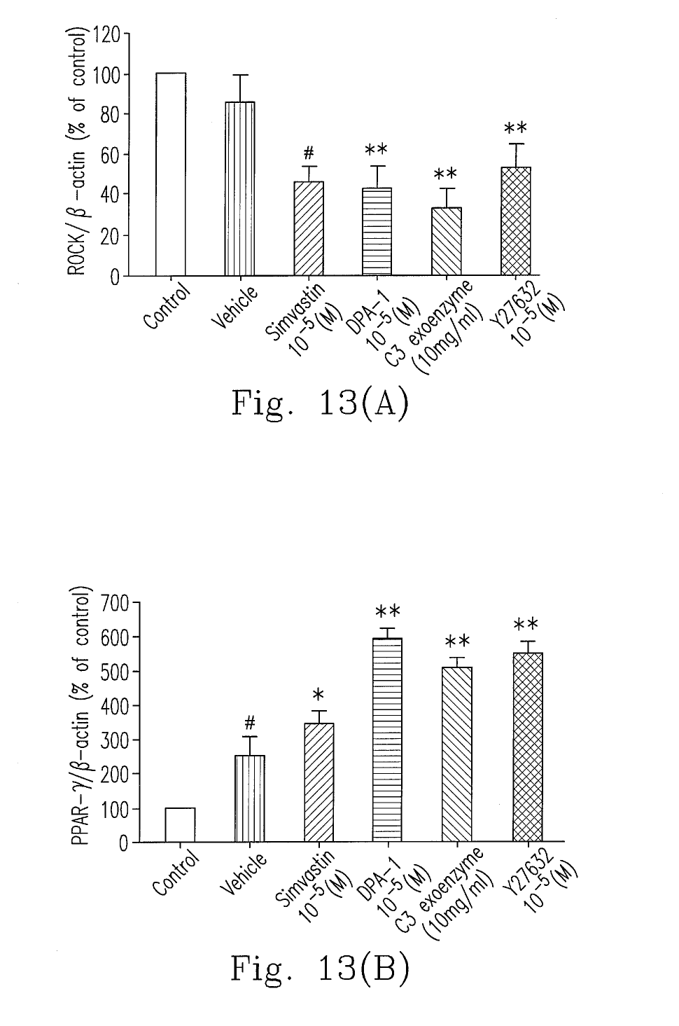

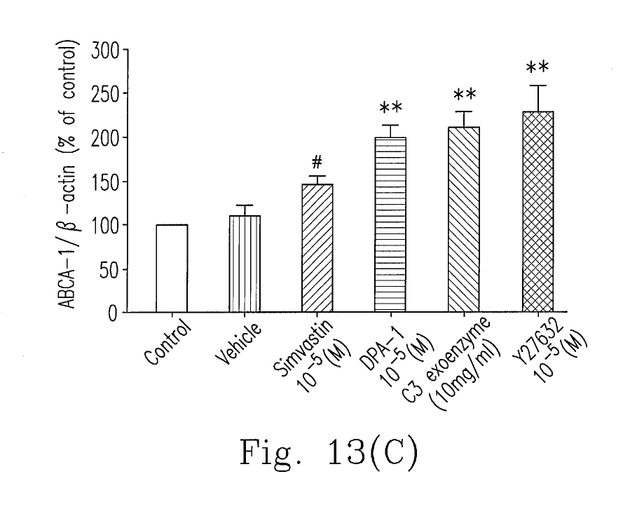

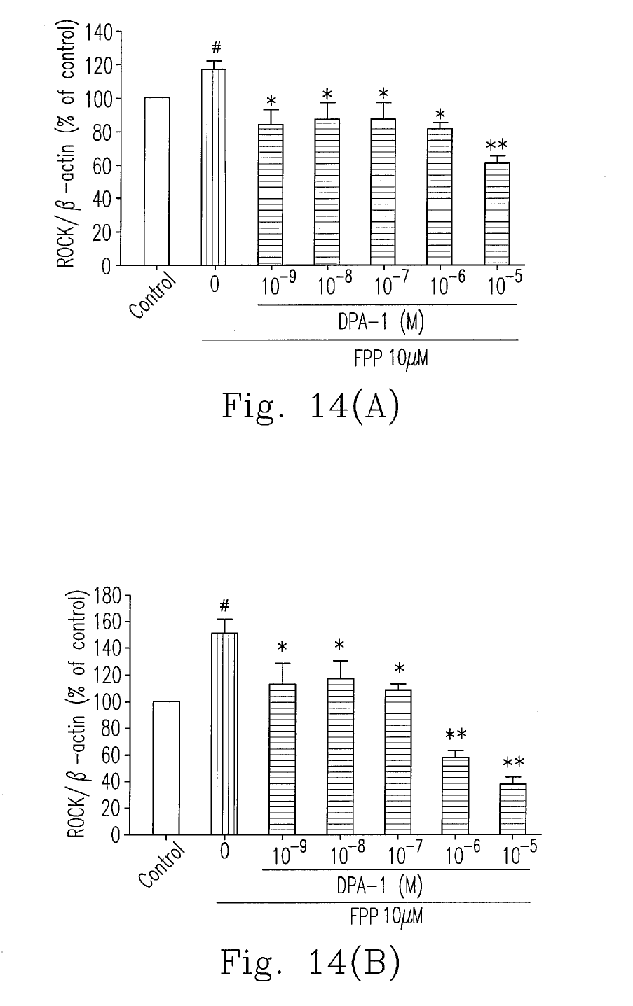

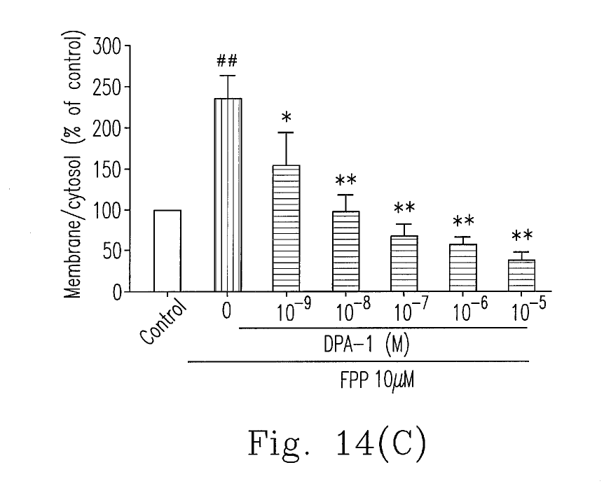

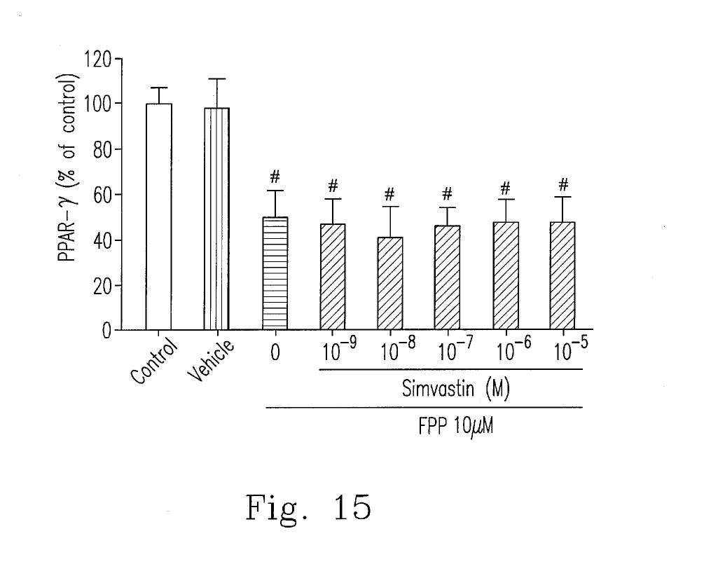

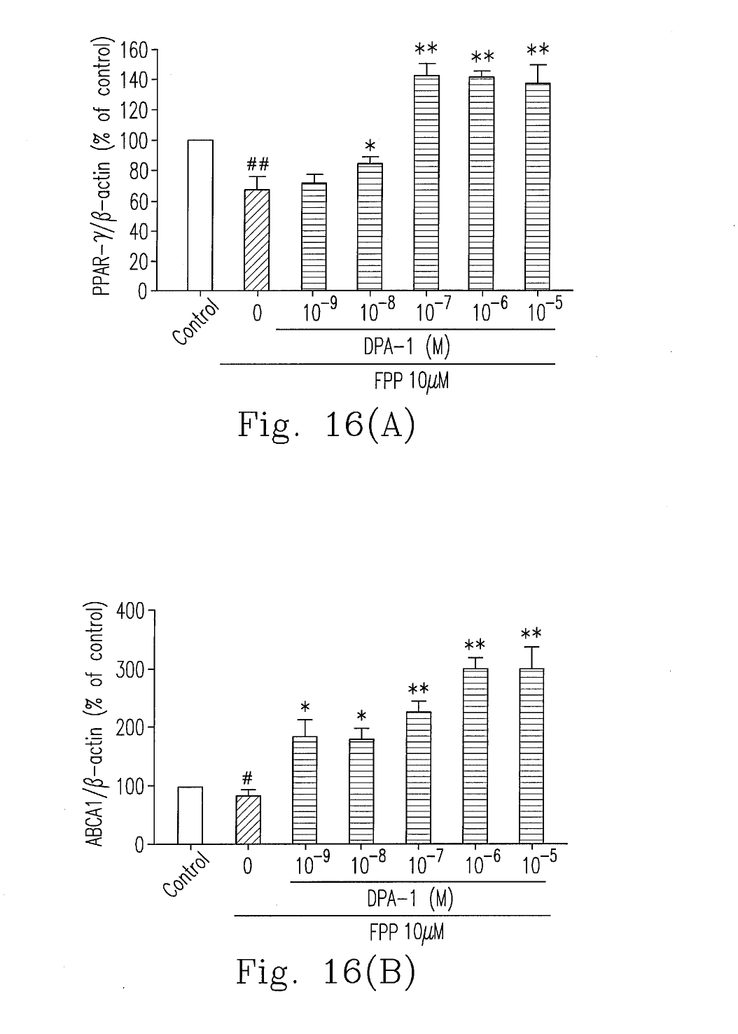

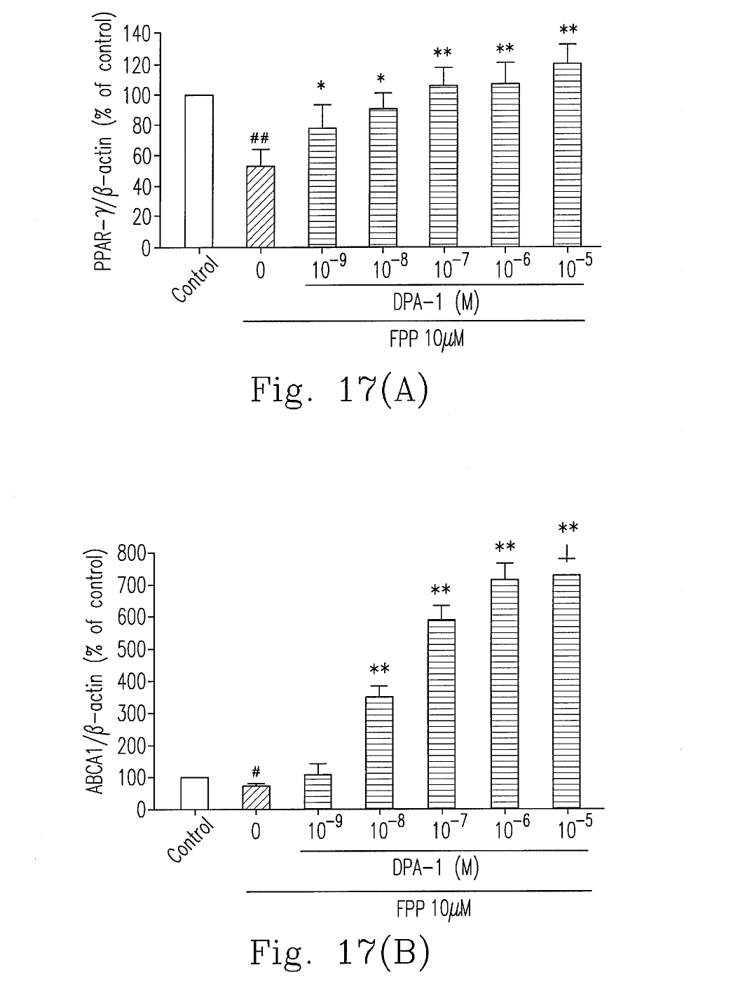

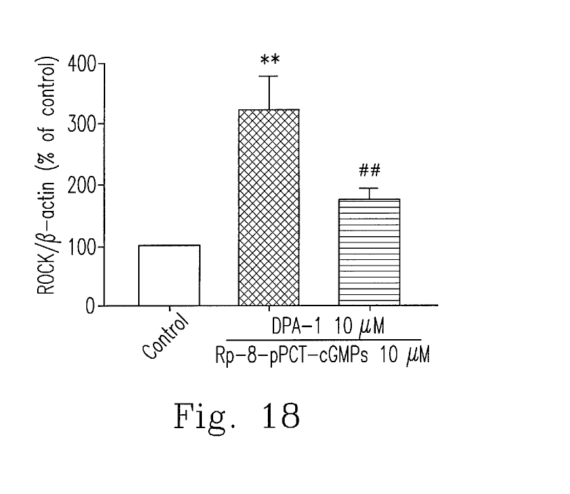

HepG2 cells were incubated with GGPP (10 μM) or FPP (10 μM) alone and GGPP or FPP with DPA-1 (10⁻⁹-10⁻⁵M) for 24 h. FIG. 9 shows the effects of DPA-1 on PPARγ, ABCA1 and in the presence of FPP and GGPP. HepG2 cells were incubated With DPA-1 (10⁻⁹-10⁻⁵M) for 24 h. FIG. 10 shows the effects of DPA-1 on LXR-α. FIG. 11 shows the effects of DPA-1 on eNOS expression. DPA-1 concentration-dependently increases eNOS expression of HePG2 cells.

Reversal of GGPP- and FPP-induced inhibition of RhoA/ROCKII and PPAR-γ/ABCA1

Exogenous application of GGPP (geranylgeranyl pyrophosphate) and FPP (farnesyl pyrophosphate) increased RhoA/ROCKII expression, but DPA-1 (10⁻⁹-10⁻⁵ M) reversed this phenomenon in HepG2 cells. Incubation of cells with FPP or GGPP (10 μM) alone decreased the expression of PPAR-γ and ABCA1, respectively. However, incubation of FPP or GGPP with DPA-1 (10⁻⁹-10⁻⁵M), reversed the reduction of PPAR-γ and ABCA1, respectively.

Pretreatment with C3 exoenzyme, Y27632, Rp-8-pPCT-cGMPs

RhoA antagonist C3 exoenzyme (5 μg/ml), ROCK antagonist Y27632 (10 μM) and cGMP antagonist Rp-8-pPCT-cGMPs (10 μM) all increased ROCKII expression but were reversed by DPA-1 (10 μM) in HepG2 cells.

TABLE 3
Effects of DPA-1 and simvastatin on HFD-
induced food intake and body weight gain

| | food-intake | body-weight | | |
|---|---|---|---|---|
| | (g/day) | Initial (g) | Final (g) | Gain (g) |
| STD | 4.0 ± 0.2 | 21.1 ± 0.5 | 24.1 ± 0.5 | 3.0 ± 0.4 |
| HFD | 2.4 ± 0.1^{#} | 22.1 ± 0.8 | 29.1 ± 0.9^{#} | 6.9 ± 0.7^{#} |
| B-1 | 2.3 ± 0.1^{#} | 22.0 ± 0.3 | 25.7 ± 0.7* | 3.7 ± 0.5* |
| C-1 | 2.2 ± 0.1^{#} | 21.2 ± 0.7 | 24.3 ± 0.5* | 3.1 ± 0.6* |
| HFD + | 2.1 ± 0.1^{#} | 21.1 ± 0.8 | 23.9 ± 0.9* | 2.8 ± 0.3* |
| simvastatin | | | | |

(note)
Data are mean ± SEM values (n = 6).
STD = standard diet fed group;
HFD = high fat diet fed group;
B-1 = high fat diet supplemented with 2.5 mg/kg DPA-1;
C-1 = high fat diet supplemented with 5 mg/kg DPA-1;
HFD + Simvastatin = high fat diet supplemented with 5 mg/kg simvastatin.
^{#}P < 0.05 vs standard diet;
*P < 0.05 vs HFD.

Effects on Body Weight Gain and Food Intake

Table 3 shows the body weight gain and food intake of animals fed with the experimental diets. Consumption of HFD (high fat diet fed group) for 8 weeks significantly increased food intake and body weight gain (p<0.05). The body weight gain of HFD was significantly increased by 2.3-fold compared to CTL (control) group (p<0.05). Surprisingly, DPA-1 (1, 2.5, 5 mg/kg p.o.) supplementation did not affect food intake in HFD fed mice. However, supplementation of DPA-1 in the HFD group significantly reduced the body weight gain compared to HFD group (p<0.05). In comparison, simvastatin and DPA-1-Simvastatin acid also affected body weight.

TABLE 4
Effects of plasma lipids by treatments

| (mg/dl) | TC | TG | HDL | LDL |
|---|---|---|---|---|
| STD | 107.2 ± 6.1 | 78.7 ± 1.9 | 60.4 ± 1.6 | 6.0 ± 0.3 |
| HFD | 166.8 ± 5.3^{#} | 206.8 ± 13.4^{##} | 68.4 ± 3.5^{#} | 31.3 ± 7.0^{##} |
| B-1 | 74.5 ± 5.1* | 133.0 ± 5.1* | 103.6 ± 4.2* | 14.2 ± 1.4* |
| C-1 | 72.7 ± 4.7* | 125.5 ± 9.8* | 118.3 ± 5.7* | 14.2 ± 2.2* |
| C-1-Sim. | 84.4 ± 45.3* | 139.75 ± 1.1* | 108.35 ± 1.3* | 14.3 ± 1.2* |
| HFD + Sim. | 82.7 ± 6.3* | 133.7 ± 4.3* | 103.2 ± 2.5* | 15.3 ± 1.3* |

(note)
Data are mean ± SEM values (n = 6).
STD = standard diet fed group;
HFD = high fat diet fed group;
B-1 = high fat diet supplemented with 2.5 mg/kg DPA-1;
C-1 = high fat diet supplemented with 5 mg/kg DPA-1;
C-1 Sim. = high fat diet supplemented with 5 mg/kg DPA-1-Simvastatin Acid;
HFD + Sim. = high fat diet supplemented with 5 mg/kg simvastatin.
^{##}P < 0.01,
^{#}P < 0.05 vs STD;
*P < 0.05 vs HFD.
HDL, High-density lipoprotein;
LDL, low-density lipoprotein
TC = total cholesterol,
TG = Triglyceride,
HDL = high density lipoprotein,
LDL = low density lipoprotein

TABLE 5
plasma levels of C57BL/6J

| (mg/dl) | TG | Tot.-C | HDL-C | LDL-C |
|---|---|---|---|---|
| STD | 107.2 ± 6.1 | 78.7 ± 1.9 | 60.5 ± 1.6 | 6 ± 0.3 |
| HFD | 166.8 ± 5.3 | 166.8 ± 13.4^{#} | 68.4 ± 3.5^{#} | 31.3 ± 7 |
| B-1 HCl | 74.5 ± 5.1* | 133 ± 5.1* | 103.6 ± 4.2* | 14.2 ± 1.4* |
| C-1 HCl | 72.67 ± 4.7* | 125.5 ± 9.8* | 118.3 ± 5.7* | 14.2 ± 2.2* |
| Sim. | 82.67 ± 6.3* | 133.6 ± 4.3* | 103.2 ± 2.5* | 15.3 ± 1.3* |
| 5 mg/kg | | | | |
| C-1 HA | 80.75 ± 7.9* | 130 ± 4.1* | 97.98 ± 3.4* | 13.8 ± 1.2* |
| B-2 HA | 79.6 ± 7.9* | 131 ± 3.5* | 99.38 ± 3.7* | 12.8 ± 1.2* |
| C-1 Sim. | 81.2 ± 4.4* | 129.7 ± 1.4* | 108.4 ± 1.3* | 14.3 ± 1.2* |
| B-1 PAA | 83.2 ± 5.3* | 129.7 ± 1.6* | 108.4 ± 1.3* | 16.4 ± 1.7* |
| B-1-γ- | 78.2 ± 3.6* | 129.4 ± 1.4* | 97.3 ± 1.6* | 16.5 ± 0.9* |
| PGA | | | | |
| B-1-CMC | 74.4 ± 4.5* | 129.7 ± 1.1* | 108.3 ± 1.3* | 16.8 ± 1.2* |
| B-1 pga | 76 ± 4.7* | 131.4 ± 1.4* | 107.5 ± 1.2* | 16.4 ± 1.1* |

(note)
Sim. (simvastatin).
B-1 CMC (DPA-1-carboxyl methylcellulose 2.5 mg/kg)
B-1 HCl (DPA-1-HCl 2.5 mg/kg)
B-1 PAA (DPA-1-polyacrylic acid 2.5 mg/kg)
B-1 pga (DPA-1-polyglycolic acid 2.5 mg/kg)
B-1 γ-PGA (DPA-1-poly-γ-polyglutamic acid 2.5 mg/kg
B-2 HA (DPA-2-hyaluronic acid 2.5 mg/kg)
C-1 Sim. (DPA-1-Simvastatin Acid 5 mg/kg)
C-1 HA (DPA-1-hyaluronic acid 5 mg/kg)
C-1 HCl (DPA-1-HCl 5 mg/kg)

Effects on Plasma Lipid Profiles

Table 5 shows the plasma TG, total cholesterol, HDL cholesterol and LDL cholesterol levels of experimental animals. In this study, the HFD caused dramatic increases in serum TG, total cholesterol and LDL cholesterol compared with the non-HFD group, by 1.6-, 2.6- and 5.2-fold, respectively. The HFD-induced hypercholesterolemia was significantly improved by DPA-1 supplementation. The HFD+DPA-1 (1, 2.5, 5 mg/kg p.o.) group had significantly reduced plasma TG (Triglyceride), total cholesterol and LDL cholesterol, compared with the HFD group. Particularly, the HDL (low-density lipoprotein) cholesterol level was significantly increased by DPA-1 (1, 2.5, 5 mg/kg p.o.) and simvastatin (5 mg/kg p.o.); the HDL value caused by DPA-1 (2.5 mg/kg) almost equal to that caused by simvastatin (5 mg/kg), indicating DPA-1 with 2 fold potency of simvastatin for increasing HDL. The LDL-lowering effects of HFD+DPA-1-Simvastatin Acid (5 mg/kg, p.o.) were located between HFD+DPA-1 (5 mg/kg, p.o.) and HFD+simvastatin (5 mg/kg, p.o.).

TABLE 6
HFD-induced body-weight growth changes and treatments with DPAs

| week | | HFD supplemented with DPA-1 | | |
|---|---|---|---|---|
| No. | HFD | A group | B group | C group |
| 10 | 23.6 ± 4.1 | 23.6 ± 3.2 | 23.8 ± 2.8 | 23.8 ± 1.9 |
| 11 | 25.2 ± 3.3 | 24.4 ± 2.6 | 25.2 ± 2.5 | 24.7 ± 3.5 |
| 12 | 28.3 ± 2.8 | 25.6 ± 2.8 | 25.3 ± 2.6 | 25.4 ± 2.4 |
| 13 | 28.4 ± 1.9 | 26.4 ± 3.7 | 28.3 ± 2.8 | 27.8 ± 1.6 |
| 14 | 30.2 ± 2.2 | 26.8 ± 1.9 | 29.4 ± 3.8 | 30.3 ± 2.9 |
| 15 | 32.4 ± 2.4 | 27.2 ± 1.6 | 32.2 ± 2.8 | 32.2 ± 2.8 |
| 16 | 34.1 ± 3.8 | 27.8 ± 2.7 | 34.6 ± 2.9 | 35.2 ± 3.4 |
| 17 | 36.4 ± 3.6 | 28.2 ± 3.3 | 36.5 ± 2.8 | 36.6 ± 2.6 |
| 18 | 50.2 ± 3.4 | 28.4 ± 3.1 | 35.4 ± 3.1 | 35.2 ± 2.1 |
| 19 | 52 ± 2.5 | 29.6 ± 2.8 | 34.7 ± 1.7 | 34.7 ± 2.4 |
| 20 | 53.6 ± 3.4 | 29.4 ± 1.8 | 33.6 ± 2.9 | 33.6 ± 2.1 |
| 21 | 54.2 ± 4.2 | 29.3 ± 2.7 | 32.6 ± 3.6 | 31.6 ± 3.8 |
| 22 | 56.1 ± 5.1 | 29.6 ± 3.5 | 31.4 ± 2.8 | 31.4 ± 2.5 |
| 23 | 58.1 ± 3.8*# | 28.2 ± 2.7# | 30.2 ± 2.6# | 29.2 ± 2.5# |

| week | HFD supplemented with DPA-2 | | | HFD supplemented with DPA-3 | | |
|---|---|---|---|---|---|---|
| No. | A group | B group | C group | A group | B group | C group |
| 10 | 23.4 ± 2.2 | 23.7 ± 2.4 | 24.1 ± 2.8 | 22.8 ± 3.5 | 23.4 ± 2.6 | 24.6 ± 3.2 |
| 11 | 24.4 ± 2.6 | 25.1 ± 2.5 | 25.2 ± 2.5 | 24.4 ± 2.6 | 25.3 ± 2.5 | 25.4 ± 2.5 |
| 12 | 25.3 ± 1.8 | 25.3 ± 2.6 | 25.3 ± 2.6 | 24.6 ± 2.6 | 25.3 ± 2.6 | 25.3 ± 2.6 |
| 13 | 26.4 ± 3.7 | 28.3 ± 2.8 | 28.3 ± 2.8 | 26.4 ± 3.7 | 28.3 ± 2.8 | 28.3 ± 2.8 |
| 14 | 27.6 ± 1.9 | 29.4 ± 3.8 | 29.4 ± 3.8 | 27.6 ± 1.9 | 29.4 ± 3.8 | 29.4 ± 3.8 |
| 15 | 28.6 ± 1.6 | 32.2 ± 2.8 | 32.2 ± 2.8 | 28.6 ± 1.6 | 32.2 ± 2.8 | 32.2 ± 2.8 |
| 16 | 29.7 ± 2.7 | 34.6 ± 2.9 | 34.6 ± 2.9 | 29.7 ± 2.7 | 34.6 ± 2.9 | 34.6 ± 2.9 |
| 17 | 30.7 ± 4.2 | 36.2 ± 2.3 | 36.7 ± 2.4 | 30.4 ± 3.3 | 36.5 ± 3.7 | 36.5 ± 2.7 |
| 18 | 31.6 ± 3.4 | 35.1 ± 2.6 | 35.7 ± 2.3 | 31.2 ± 2.3 | 35.4 ± 2.8 | 35.9 ± 2.4 |
| 19 | 31.6 ± 2.8 | 4.7 ± 1.7 | 34.7 ± 1.7 | 31.6 ± 2.4 | 34.4 ± 1.4 | 34.8 ± 1.6 |
| 20 | 30.6 ± 1.8 | 33.6 ± 2.5 | 33.6 ± 2.6 | 30.6 ± 1.8 | 33.6 ± 2.8 | 33.6 ± 2.8 |
| 21 | 30.5 ± 2.7 | 32.6 ± 3.6 | 32.6 ± 3.6 | 30.5 ± 2.7 | 32.6 ± 3.6 | 32.6 ± 3.6 |
| 22 | 29.6 ± 3.5 | 31.4 ± 2.8 | 31.4 ± 2.8 | 29.6 ± 3.5 | 31.4 ± 2.8 | 31.4 ± 2.8 |
| 23 | 28.1 ± 2.5# | 29.2 ± 3.5# | 29.4 ± 3.5# | 28.4 ± 3.2# | 29.2 ± 3.9# | 30.4 ± 2.5# |

| week | | HFD supplemented with DPA-4 | | |
|---|---|---|---|---|
| No. | HFD | A group | B group | C group |
| 10 | 23.6 ± 4.1 | 22.9 ± 3.7 | 24.2 ± 1.6 | 23.2 ± 1.8 |
| 11 | 25.2 ± 3.3 | 24.8 ± 3.1 | 25.2 ± 2.5 | 25.1 ± 2.3 |
| 12 | 28.3 ± 2.8 | 24.8 ± 2.5 | 25.3 ± 2.6 | 25.3 ± 2.4 |
| 13 | 28.4 ± 1.9 | 26.4 ± 3.7 | 28.3 ± 2.8 | 28.3 ± 2.6 |
| 14 | 30.2 ± 2.2 | 27.6 ± 1.9 | 29.4 ± 3.8 | 29.4 ± 3.6 |
| 15 | 32.4 ± 2.4 | 27.7 ± 1.6 | 32.2 ± 2.7 | 32.2 ± 2.9 |
| 16 | 34.1 ± 3.8 | 28.4 ± 2.7 | 34.6 ± 2.9 | 34.6 ± 3.1 |
| 17 | 36.4 ± 3.6 | 28.6 ± 3.1 | 35.9 ± 3.8 | 36.6 ± 2.7 |
| 18 | 50.2 ± 3.4 | 29.2 ± 3.1 | 35.1 ± 2.6 | 35.7 ± 2.5 |
| 19 | 52 ± 2.5 | 30.6 ± 2.8 | 34.7 ± 1.5 | 34.5 ± 1.7 |
| 20 | 53.6 ± 3.4 | 30.9 ± 2.3 | 33.6 ± 2.8 | 33.6 ± 2.8 |
| 21 | 54.2 ± 4.2 | 29.5 ± 2.7 | 31.8 ± 3.6 | 32.6 ± 3.6 |
| 22 | 56.1 ± 5.1 | 28.8 ± 2.5 | 31.4 ± 2.8 | 31.4 ± 2.8 |
| 23 | 58.1 ± 3.8*# | 27.8 ± 2.3# | 30.2 ± 3.5# | 29.2 ± 3.6# |

(note)
Data are mean ± SEM values (n = 9).
HFD = high fat diet fed group;
HFD supplemented with DPAsA group = high fat diet supplemented with 1 mg/kg DPA-1 DPA-2, DPA-3 and DPA-4 separately, beginning at week 10 for 12 weeks experiment;
HFD supplemented with DPAsB group = high fat diet supplemented with 2.5 mg/kg DPA-1, DPA-2, DPA-3 and DPA-4 separately, beginning at 18 w for 6 weeks experiment.
HFD supplemented with DPAsC group = high fat diet supplemented with 5.0 mg/kg DPA-1, DPA-2, DPA-3 and DPA-4 separately, beginning at wee 18 for 6 weeks experiment.
Other analogues were administered according to same way.
C57BL/6J mice were sacrificed at week 23;
*P < 0.05, treatments vs HFD.
#P < 0.05, week-23 vs week-10

Table 7 shows the oral administration of HFD significantly increased body-weight growth at week 18 and at week-23. All treatments significantly reduced HDF-induced body-weight gain. The body-weight gain at week-17 is significantly different from week-10. Dose-dependent responses to 1, 2.5 and 5 mg/kg of treatment were found (P<0.05). The body-weight gain at week-23 was insignificantly decreased by treatment (A group, 1 mg/kg), compared to week-18. However, this decrease at week-23 was significantly different from that of week-18 by treatment (B group, 2.5 mg/kg) and (C group, 5 mg/kg).

TABLE 7
HFD-induced body-weight gain and treatment with DPAs

| | Body-weight (g) | | | | Total Body-weight (g) | |
|---|---|---|---|---|---|---|
| | 10 w | 17 w | 18 w | 23 w | 17 w | 23 w |
| HFD | 23.6 ± 4.1 | 36.4 ± 3.6 | 50.2 ± 3.4 | 58.6 ± 3.8 | +23.3 ± 3.6 | +8.4 ± 0.2 |
| A-1 | 23.6 ± 3.2 | 28.2 ± 3.3 | 28.4 ± 3.1 | 28.2 ± 2.7 | +6.8 ± 0.9; | −0.2 ± 0.4 |
| A-2 | 23.4 ± 2.2 | 30.7 ± 4.2 | 31.6 ± 3.4 | 28.1 ± 2.5 | +7.3 ± 1.9 | −3.5 ± 1.1 |
| A-3 | 22.8 ± 3.5 | 30.4 ± 3.3 | 31.2 ± 2.3 | 28.4 ± 3.2 | +7.6 ± 0.2 | −2.8 ± 1.1 |
| A-4 | 22.9 ± 3.7 | 30.4 ± 3.1 | 28.6 ± 2.7 | 27.8 ± 2.3 | +7.5 ± 0.5 | −0.8 ± 0.4 |
| B-1 | 23.8 ± 2.8 | 36.5 ± 2.8 | 35.4 ± 3.1 | 30.2 ± 2.6 | +2.7 ± 0.1 | −5.2 ± 0.5 |
| B-2 | 23.7 ± 2.4 | 36.2 ± 2.3 | 35.1 ± 2.6 | 29.2 ± 3.5 | +12.5 ± 0.2 | −5.9 ± 0.9 |
| B-3 | 23.4 ± 2.6 | 36.5 ± 3.7 | 35.4 ± 2.8 | 29.2 ± 3.9 | 13.1 ± 1.1 | −6.2 ± 0.1 |
| B-4 | 24.2 ± 1.6 | 35.9 ± 3.8 | 35.1 ± 2.6 | 30.2 ± 3.5 | 11.7 ± 0.1 | −4.9 ± 0.3 |
| C-1 | 23.8 ± 1.9 | 36.6 ± 2.6 | 35.2 ± 2.1 | 29.2 ± 1.5 | +12.8 ± 0.7 | −6.3 ± 0.4 |
| C-2 | 24.1 ± 2.8 | 36.7 ± 2.4 | 35.7 ± 2.3 | 29.4 ± 3.5 | +12.6 ± 0.4 | −6.3 ± 1.1 |
| C-3 | 24.6 ± 3.2 | 36.5 ± 2.7 | 35.9 ± 2.4 | 30.4 ± 2.5 | +11.9 ± 0.5 | −5.5 ± 1.1 |
| C-4 | 23.2 ± 1.8 | 36.6 ± 2.7 | 35.7 ± 2.5 | 29.2 ± 3.6 | +13.4 ± 0.9 | −6.5 ± 1.1 |

(note)
Data was obtained from n = 9 of each group, W (week)
*P < 0.05 vs HFD;
A-1 = high fat diet supplemented with 1 mg/kg DPA-1 beginning at week 10 for 12 weeks experiment;
A-2 = high fat diet supplemented with 1 mg/kg DPA-2 beginning at week 10 for 12 weeks experiment;
A-3 = high fat diet supplemented with 1 mg/kg DPA-3 beginning at week 10 for 12 weeks experiment;
A-4 = high fat diet supplemented with 1 mg/kg DPA-4 beginning at week 10 for 12 weeks experiment;
Other analogues were administered according to same way. So that B group (B-1, B-2, B-3 and B-4) and C group (C-1, C-2, C-3 and C-4) dose-dependent responses to 2.5 and 5 mg/kg DPAs of treatment separately.
C57BL/6J mice were sacrificed at week 23; *P < 0.05, treatments vs HFD. # P < 0.05, week-23 vs week-10

Table 8 shows the Feeding-Rate is the percentage of food intake during one week. Obesity mice aging 18 weeks were administered with high fat diet (HFD) and mice with similar body weight were collected for experiments. Other analogues were administered according to same way. C57BL/6J mice were sacrificed at week 23; *P<0.05 vs HFD; #P<0.05 vs week-18; DPA-2, DPA-3 and DPA-4 were shown as the same manner. They all reduced feeding rate significantly at week-23 or week 22. DPAs save the consumption of food additives dose-dependently, leading to decreasing the feeding-rate of HFD.

TABLE 8
Feeding-Rate of HFD and treatment with DPAs focused
at week 18~23 after birth

| Dosage of | Feeding-Rate (g/week) | | | | | |
|---|---|---|---|---|---|---|
| compounds | 18 | 19 | 20 | 21 | 22 | 23 |
| HFD | 29.1 ± 3.2 | 29.7 ± 2.6 | 30.4 ± 4.2 | 31.2 ± 3.7 | 32.6 ± 2.8 | 33.3 ± 1.9 |
| A-1 | 28.7 ± 2.8 | 27.4 ± 2.6 | 28.1 ± 1.9 | 27.9 ± 2.3 | 26.4 ± 2.5 | 24.2 ± 1.8* |
| B-1 | 28.5 ± 2.6 | 27.5 ± 1.8 | 25.3 ± 2.1 | 24.2 ± 1.7 | 23.4 ± 2.2* | 23.1 ± 1.8* |
| C-1 | 28.4 ± 2.1 | 26.8 ± 2.4 | 25.6 ± 2.6 | 24.4 ± 2.5 | 23.1 ± 1.8* | 21.8 ± 2.1* |
| A-2 | 28.6 ± 2.2 | 28.5 ± 2.4 | 28.3 ± 1.5 | 27.6 ± 1.8 | 26.5 ± 2.4 | 25.1 ± 2.6* |
| B-2 | 28.4 ± 1.9 | 27.1 ± 2.3 | 25.9 ± 1.8 | 24.4 ± 2.5 | 23.2 ± 2.2* | 22.8 ± 1.9* |
| C-2 | 28.5 ± 2.0 | 26.3 ± 1.9 | 25.5 ± 1.8 | 24.1 ± 2.3 | 22.8 ± 1.7* | 21.7 ± 2.5* |
| A-3 | 28.3 ± 1.7 | 28.5 ± 1.6 | 27.6 ± 2.4 | 28.1 ± 2.1 | 26.4 ± 2.4 | 24.3 ± 1.8* |
| B-3 | 28.2 ± 2.3 | 28.3 ± 1.9 | 28.4 ± 2.3 | 23.2 ± 1.8* | 22.8 ± 2.3* | 23.5 ± 2.7* |
| C-3 | 28.4 ± 2.1 | 26.3 ± 1.9 | 25.1 ± 1.7 | 23.6 ± 2.6* | 22.4 ± 2.7* | 22.8 ± 2.8* |
| A-4 | 29.3 ± 2.4 | 28.3 ± 2.6 | 28.1 ± 2.7 | 27.4 ± 1.9 | 26.7 ± 2.2* | 24.6 ± 2.1* |
| B-4 | 29.2 ± 2.5 | 28.6 ± 2.4 | 25.4 ± 1.8* | 24.5 ± 2.3* | 23.7 ± 1.8* | 22.3 ± 1.5* |
| C-4 | 29.4 ± 1.7 | 28.2 ± 2.3 | 28.5 ± 2.4 | 23.1 ± 1.9* | 22.6 ± 1.6* | 22.5 ± 1.7* |

(note)
Data was obtained from n = 9 of each group,
Data are mean ± SEM values (n = 9).
HFD = high fat diet fed group;
A-1 = high fat diet supplemented with 1 mg/kg DPA-1 beginning at week 10 for 14 weeks experiments;
B-1 = high fat diet supplemented with 2.5 mg/kg DPA-1 beginning at week 18 for 6 weeks experiments;
C-1 = high fat diet supplemented with 5.0 mg/kg DPA-1 beginning at week 18 for 6 weeks experiments;
According that A-2, B-2 and C-2 supplemented with DPA-2; A-3, B-3 and C-3 supplemented with DPA-3; A-4, B-4 and C-4 supplemented with DPA-4.
C57BL/6J mice were sacrificed at week 23; *P < 0.05 vs HFD; #P < 0.05 vs week-18.

Table 9 shows DPAs-treatments reduced the values of ratio, indicating inhibiting the development of adipose % of body fats

TABLE 9
Two epididymal fat pads-weight/body-weight ratio
of HFD and treatments with DPAs for 6 weeks

| | Two epididymal fat | Body-weight (g) | |
|---|---|---|---|
| | pad-weight (g) | (at week-23) | Ratio |
| HFD | 0.5039 ± 0.0511 | 58.6 ± 3.8** | 0.86 |
| A-1 | 0.2115 ± 0.0312 | 28.2 ± 2.7^{#} | 0.75 |
| B-1 | 0.21442 ± 0.003 | 30.2 ± 2.6^{#} | 0.71 |
| C-1 | 0.19272 ± 0.002 | 29.2 ± 1.5^{#} | 0.66 |
| A-2 | 0.21637 ± 0.0407 | 28.1 ± 2.5^{#} | 0.77 |
| B-2 | 0.21316 ± 0.004 | 29.2 ± 3.5^{#} | 0.73 |
| C-2 | 0.19272 ± 0.003 | 29.2 ± 1.5^{#} | 0.66 |
| A-3 | 0.22152 ± 0.004 | 28.4 ± 3.2^{#} | 0.78 |
| B-3 | 0.21608 ± 0.005 | 29.2 ± 3.2^{#} | 0.74 |
| C-3 | 0.20064 ± 0.004 | 30.4 ± 2.5^{#} | 0.66 |

(note)
Data are mean ± SEM values (n = 9).
HFD = high fat diet fed group;
A-1 = high fat diet supplemented with 1 mg/kg DPA-1 beginning at week 10 for 12 weeks;
B-1 = high fat diet supplemented with 2.5 mg/kg DPA-1 beginning at week-18 for 4 weeks experiments;
C-1 = high fat diet supplemented with 5.0 mg/kg DPA-1 beginning at week-18 for 6 weeks experiments.
Other analogues were administered according to same way, A-2, B-2 and C-2 supplemented with DPA-2; A-3, B-3 and C-3 supplemented with DPA-3.
C57BL/6J mice were sacrificed at week-23;
**P < 0.01 treatments of DPAs vs HFD.
Adminidtration of DPAs for 6 weeks dose-dependently inhibited HFD-induced ratio of “two epididymalfat pad-weight (g)/body-Weight (g)”, indicating increasing the lipolysis activity of adipose tissue by DPAs.

Anti-Atherosclerosis

HFD induced morphological changes, including narrowing the vascular wall thickness. Treatment with DPA-1 dose-dependently prevent from the narrowing. As shown in Table 10, DPAs dose-dependently reduced the wall thickness.

TABLE 10
Effects on wall thickness and treatment with DPAs

| Treatment (p.o.) | Wall thickness % |
|---|---|
| STD | 26.9 ± 2.7 |
| HFD | 48.9 ± 8.7 ^{#} |
| A-1 | 35.3 ± 2.4* |
| B-1 | 32.3 ± 1.8* |
| A-2 | 36.2 ± 2.4* |
| B-2 | 31.3 ± 1.8* |
| A-3 | 38.2 ± 3.6* |
| B-3 | 34.3 ± 2.7* |

(note)
Values are means ± SE of n = 6.
*P < 0.05 vs HFD;
^{#} P < 0.05 vs STD (control);
DPAs dose-dependently reduced the aorta thickness, compared to HDF group.

Expression/Activity of HMGR

To demonstrate that DPA-1, simvastatin and DPA-1-Simvastatin Acid can affect HMGR (HMG-CoA reductase), we measured the HMGR protein expression and activity in HepG2 cell. Incubation of HepG2 cells with them for 24 h, significantly enhanced HMGR protein expressions. DPA-1, simvastatin and DPA-1-Simvastatin Acid (10⁻⁹-10⁻⁵ μM) all concentration-dependently inhibited NADPH, the represented HMGR activity. However, supplementation of DPA-1 (2.5-5 mg/kg) and simvastatin (5 mg/kg) in the HFD group significantly reduced the HMGR reductase.

The term excipients or “pharmaceutically acceptable carrier or excipients” and “bio-available carriers or excipients” above-mentioned include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-avaliable enhancing agent used for developing the formulation that used in the pharmaceutical industry.

The composition for oral administration adopts any oral acceptable formulation, which includes capsule, tablet, pill, emulsion, aqueous suspension, dispersing agent and solvent. The carrier generally used in the oral formulation, taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

Animal Model of Hyperlipidemia

C57BL/6J mice were fed with high-fat diet (HFD) to produce a model of hyperlipidemia. At 6-week old, the C57BL/6J mice were randomly divided into 5 groups, two control and three treatment groups. The control mice received either a regular diet or HFD and the treatment group was fed with a HFD and DPA-1 (1, 2.5, 5 mg/kg, p.o.), or simvastatin (5 mg/kg), respectively, for a period of 8 weeks. Compared to two control groups, the body-weight gain and lipid levels were measured in HFD group. In the measurement of eNOS and ROCKII expression of liver, mice (20˜25 g) were administered with DPASor simvastatin and sacrificed to obtain liver at 1 and 24 hours.

Animal Feeding via HFD and Treatment with DPAs on Mice Body-Weight Gain

Male C57BL/6J mice (aging 10 weeks) were housed one-mouse to each-cage fully loaded with 50 g diet, maintained at 25° C. on a 12-h light-dark cycle, and provided high-fat diet (HFD) and water ad libitium. The care of mice, as well as all procedure used in this study, were done in accordance with National Institutes of Health animal care guidelines. Consumed or fed diet, as food-intake was calculated as a reference of feeding rate.

Serum Cholesterol and Lipids

Mice serum was collected by centrifugation. Serum triglyceride, cholesterol, LDL and HDL levels were measured using a Hitachi Clinical Analyzer 7070 (Hitachi High-Technologies Co. Tokyo, Japan).

Western Blotting Analyses of Proteins in 3-T-3 Cell Culture

The 3T3-L1 adipocytes in 100 mm dishes were stimulated with MDI and DPA-1 for the indicated time, and whole cell extracts were prepared by lysing the cells in extraction buffer (RIPA buffer) containing 50 mmol/L Tris(hydroxymethyl)aminomethane (Tris)/HCl, pH 8.0, 150 mmol/L NaCl, 1% Nonidet-P40, 1% sodium deoxycholate, 0.1% sodium dodecyl sulfate (SDS), 0.1 mmol/L dithiothreitol (DTT), 0.05 mmol/L phenylmethyl-sufonyl-fluoride (PMSF), 0.002 mg/mL aprotinin, 0.002 mg/mL leupeptin and 1 mmol/L NaVO3 after stimulation. The protein concentration was quantified with Bio-Rad Dc protein assay reagent (Bio-Rad, Hercules, USA). Equal amounts of protein were mixed with SDS sample buffer and incubated for 5 min at 100° C. before loading. Sodium dodecyl sulfate-polyacrylamide gel electrophoresis (SDS-PAGE) was performed according to the method of Amersham Biosciences. Immunoreactive bands were detected by means of an ECL plus western blotting detection system (Amersham Biosciences). The chemiluminescent signals were scanned from films (Nippon Polaroid K.K., Tokyo, Japan).

Protein Expression by Western Blotting Analysis of Mice Liver and HepG2 Cells

Liver tissues of mice were isolated and cut into small chips and HepG2 cell were placed into an extraction buffer (Tris 10 mM, pH 7.0, NaCl 140 mM, PMSF 2 mM, DTT 5 mM, NP-40 0.5%, pepstatin A 0.05 mM and leupeptin 0.2 mM) for protein extraction, and then centrifuged at 12,500 g for 30 min, respectively. To measure the expression levels of proteins, the total proteins were extracted and measured the protein by Western blotting analyses. Briefly, the protein extract was boiled to a ratio of 4:1 with sample buffer (Tris 100 mM, pH 6.8, glycerol 20%, SDS 4% and bromophenol blue 0.2%). Electrophoresis was performed using 10% SDS-polyacrylamide gel (2 hr, 100 V, 40 mA, 50 mg protein per lane). Separated proteins were transferred to PVDF membranes treated with 5% fat-free milk powder to block the nonspecific IgGs (90 min, 100 V) and incubated for 1 hr with specific protein antibody. The blot was then incubated with anti-mouse or anti-goat IgG linked to alkaline phosphatase (1:1000) for 1 hr. Immunoreactive bands were visualized using horseradish peroxidase-conjugated secondary antibodies and subsequent ECL detection (GE Healthcare Bio-Sciences Corp., Piscataway, N.J., U.S.A.). Mouse or rabbit monoclonal antibody of ROCKII (Upstate, N.Y., U.S.A.), RhoA (Santa Cruze, Calif., U.S.A.), HMG CoA reductase (Upstate, N.Y, U.S.A.), PPAR-γ (Santa Cruze, Calif., U.S.A.), ABCA1 (Cell Signaling, NY, U.S.A.), APOA-1 (Abcam, N.Y., U.S.A.), LXR (Santa Cruze, Calif., U.S.A.) and the loading control protein β-actin (Sigma-Adrich, MO) were used in our Western blot analyses.

### Embodiments

### Example 1

### Preparation of DPA-1 HCl

2-Chloroethyl theophylline (8.3 g), NaOH (8.3 g) and 2-chlorobenzene-piperazine (8.3 g) are dissolved in hydrous ethanol (100 mL) and then heated under reflux for 3 h. Allowed to stand overnight, the cold supernatant was decanted for processing, efficient removal of solvents by vacuum concentration, and then the residue were dissolved with one-fold volume of ethanol and three-fold volume of 2N hydrochloric acid (HCl), kept at room temperature, to make a saturated solution (pH 1.2). The saturated solution was sequentially treated, decolorized with activated charcoal, filtered, deposited overnight and filtered to obtain DPA-1 HCl with a white crystal.

### Example 2

### Preparation of DPA-1-CMC Salt

Method 1: 20 g Sodium carboxyl methylcellulose is suspended in distill water and added with DPA-1 HCl (16 g) and methanol 100 ml to reflux in a three-neck reactor, equiped with a condenser, for 1 hour. After cooling, obtained precipitate is dissolved in methanol 100 ml and the resulted solution is incubated for crystallization and filtrated to obtain DPA-1-CMC salt (35.4 g).

Method 2: DPA-1 HCl (16 g) is dissolved in methanol (100 ml) and added with sodium CMC (20 g) and refluxed in a three-neck reactor, equiped with a condenser, for 1 hour. After cooling, obtained precipitate is filtrated and re-crystallized with methanol 100 ml to have DPA-1-CMC salt (35.2 g).

### Example 3

### Preparation of DPA-1-γ-Polyglutamic Acid

Method 1: Sodium γ-polyglutamic acid (20 g) is suspended in distill water and added with DPA-1 HCl (16 g) dissolved in methanol 100 ml to reflux in a three-neck reactor, equiped with a condenser, for 1 hour. After cooling, obtained precipitate is dissolved in methanol 100 ml and the resulted solution is incubated for crystallization and filtrated to obtain DPA-1-γ-Polyglutamic acid (35.6 g).

Method 2: DPA-1 HCl (16 g) is dissolved in methanol 100 ml and added with sodium alginic acid 20 g dissolved in methanol 100 ml to reflux in a three-neck reactor, equiped with a condenser, for 1 hour. After cooling, obtained precipitate is filtrated and re-crystallized with methanol 100 ml to have DPA-1-γ-Polyglutamic acid (35. 8 g).

### Example 4

### Preparation of DPA-1-Alginic Acid

Method 1: Sodium alginic acid (20 g) is suspended in distill water and added with DPA-1 HCl (16 g) dissolved in and methanol 100 ml to reflux in a three-neck reactor, equiped with a condenser, for 1 hour. After cooling, obtained precipitate is dissolved in methanol 100 ml and the resulted solution is incubated for crystallization and filtrated to obtain DPA-1-Alginic acid (35.4 g).

Method 2: DPA-1 HCl 16 g is dissolved in methanol 100 ml, added with sodium alginic acid 20 g dissolved in methanol 100 ml to reflux in a three-neck reactor, equiped with a condenser, for 1 hour. After cooling, obtained precipitate is filtrated and re-crystallized with methanol 100 ml to have DPA-1-Alginic acid (35.6 g).

### Example 7

### Preparation of DPA-2-polyacrylic Acid

DPA-2 HCl (8 g) is dissolved in a mixture of methanol (100 mL) and sodium polyacrylic acid (2.5 g). The solution is refluxed in a three-neck reactor, equiped with a condenser, for 1 hour. After cooling, obtained precipitate is re-dissolved in methanol 100 ml and the resulted solution is incubated for crystallization and filtrated to obtain DPA-2-polyacrylic acid (7.4 g).

### Example 8

### Preparation of DPA-2-γ-Polyglutamic Acid

DPA-2 HCl (8 g) is dissolved in a mixture of methanol (100 mL) and sodium γ-Polyglutamic acid (2.5 g). The solution is refluxed reflux in a three-neck reactor, equiped with a condenser, for 1 hour. After cooling, obtained precipitate is re-dissolved in methanol 100 ml and the resulted solution is incubated for crystallization and filtrated to obtain DPA-2-γ-Polyglutamic acid (10.3 g).

### Example 9

### Preparation of DPA-1-dextran Sulfate Salt

DPA-1 HCl (8 g) is dissolved in a mixture of methanol (100 mL) and sodium dextran sulfate (3.5 g). The solution is refluxed reflux in a three-neck reactor, equiped with a condenser, for 1 hour. After cooling, obtained precipitate is re-dissolved in methanol 100 ml and the resulted solution is incubated for crystallization and filtrated to obtain DPA-1-dextran sulfate salt (10.6 g).

### Example 10

### Preparation of DPA-4-heparan Sulfate Salt

DPA-4 HCl (8.3 g) is dissolved in a mixture of methanol (100 mL) and sodium heparan sulfate (2.5 g). The solution is refluxed in a three-neck reactor, equiped with a condenser, for 1 hour. Obtained precipitate is re-dissolved in methanol 100 ml and the resulted solution is incubated for crystallization and filtrated to obtain DPA-4-heparan sulfate salt (9.2 g).

### Example 11

### Preparation of DPA-2-hyaluronic Acid

DPA-2 HCl (8 g) is dissolved in a mixture of methanol (100 mL) and sodium hyaluronic acid (2.5 g). The solution is refluxed in a three-neck reactor, equiped with a condenser, for 1 hour. After cooling, obtained precipitate is re-dissolved in methanol 100 ml and the resulted solution is incubated for crystallization and filtrated to obtain DPA-2-hyaluronic acid (9.4 g).

### Example 12

### Preparation of DPA-1-Simvastatin Complex

DPA-1 (8.0 g) is dissolved in a mixture of ethanol (10 mL) and HCl (1 N, 60 mL) and reacted at 50° C. for 10 min, the methanol is added thereinto under room temperature and the solution is incubated over night for crystallization and filtrated to obtain DPA-1 HCl (7.4 g). Take DPA-1 HCl salt (4.4 g) and redissolve it in ethanol (150 mL) for use.

In a flask equipped with a magnetic stirrer, simvastatin (4.2 g) dissolved in ethanol (50 ml) is poured, to which an aqueous solution of sodium hydroxide (4 g/60 ml) and the above-mentioned filtrate of DPA-1 HCl salt reacted with the ethanol are added under room temperature. The mixture is reacted at 50° C. for 20 mins, rapidly filtrated and incubated one hour for crystallization to give the DPA-1-Simastatin complex.

### Example 13

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.

| | |
|---|---|
| DPA-1-hyaluronic acid | 80 mg |
| Lactose | qs |
| Corn starch | qs |

### Example 14

### Preparation of the Composition in Tablet

| | |
|---|---|
| DPA-3 | 100 mg |
| Lactose | qs |
| Corn starch | qs |

There are further embodiments provided as follows.

Embodiment 1: A DPAs amine complex compound comprising a compound represented by formula II:

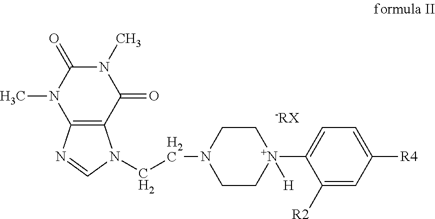

wherein: R2 and R4 are selected independently from a group consisting of a C1˜C5 alkoxy group, a hydrogen, a nitro group, and a halogen atom;
RX includes a carboxylic group selected from a group consisting of a Statin derivative, a sodium carboxyl methylcellulose (sodium CMC), a poly-γ-polyglutamic acid (γ-PGA) derivative and a co-polymer ; and
⁻RX substituent is an anion of the carboxylic group carrying a negative charge, halogen atom is one selected from a group consisting of a fluorine, a chlorine, a bromine and an iodine.

Embodiment 2: The DPAs amine complex compound of the above-mentioned embodiment, wherein the halogen atom is one selected from a group consisting of a fluorine, a chlorine, a bromine and an iodine.

Embodiment 3: The DPAs amine complex compound of one of the above-mentioned embodiments, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

Embodiment 4: The DPAs amine complex compound of any of the above-mentioned embodiments, wherein the co-polymer includes one selected from a group consisting of a hyaluronic acid a polyacrylic acid, a polymethacrylates (PMMA), an Eudragit, a dextran sulfate, a heparan sulfate, a polylactic acid (PLA), a polylactic acid sodium (PLA sodium) and polyglycolic acid sodium (pga sodium).

Embodiment 5: The DPAs amine complex compound of any one of the above-mentioned embodiments, wherein the compound comprising one selected from a group consisting of a DPA-1-RX, a DPA-2-RX, a DPA-3-RX and a DPA-4-RX.

Embodiment 6: The DPAs amine complex compound of any one of the above-mentioned embodiments, wherein the DPA-1-RX includes a 7-[2-[4-(2-chlorophenyl)piperazinyl]ethyl]-1,3-dimethylxanthine-RX.

Embodiment 7: The DPAs amine complex compound of any one of the above-mentioned embodiments, wherein the DPA-2-RX includes a 7-[2-[4-(2-methoxybenzene)piperazinyl]ethyl]-1,3-dimethylxanthine-RX.

Embodiment 8: The DPAs amine complex compound of any one of the above-mentioned embodiments, wherein the DPA-3-RX includes a 7-[2-[4-(4-nitrobenzene)piperazinyl]ethyl]-1,3-dimethylxanthine-RX.

Embodiment 9: The DPAs amine complex compound of any one of the above-mentioned embodiments, wherein the DPA-4-RX includes a 7-[2-[4-(2-nitrobenzene)piperazinyl]ethyl]-1,3-dimethylxanthine-RX.

Embodiment 10: A pharmaceutical composition comprising a DPAs amine complex compound represented by a structure being formula II,

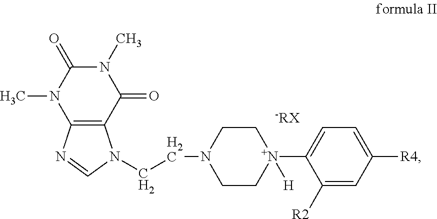

wherein: R2 and R4 are selected independently from a group consisting of a C1˜C5 alkoxy group, a hydrogen, a nitro group, and a halogen atom;
RX includes a carboxylic group selected from a group consisting of a Statin derivative, a sodium carboxyl methylcellulose (sodium CMC), a poly-γ-polyglutamic acid (γ-PGA) derivative, a co-polymer and a combination thereof; and
⁻RX substituent is an anion of the carboxylic group carrying a negative charge, halogen atom is one selected from a group consisting of a fluorine, a chlorine, a bromine and an iodine.

Embodiment 11: The DPAs amine complex compound of the above-mentioned embodiment, wherein the halogen atom is one selected from a group consisting of a fluorine, a chlorine, a bromine and an iodine.

Embodiment 12: The DPAs amine complex compound of one of the above-mentioned embodiments, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

Embodiment 13: The DPAs amine complex compound of any one of the above-mentioned embodiments, wherein the co-polymer includes one selected from a group consisting of a hyaluronic acid a polyacrylic acid, a polymethacrylates (PMMA), an Eudragit, a dextran sulfate, a heparan sulfate, a polylactic acid (PLA), a polylactic acid sodium (PLA sodium) and polyglycolic acid sodium (pga sodium).

Embodiment 14: The DPAs amine complex compound of any one of the above-mentioned embodiments, wherein the compound comprising one selected from a group consisting of a DPA-1-RX, a DPA-2-RX, a DPA-3-RX and a DPA-4-RX.

Embodiment 15: The DPAs amine complex compound of any one of the above-mentioned embodiments, wherein the DPA-1-RX includes a 7-[2-[4-(2-chlorophenyl)piperazinyl]ethyl]-1,3-dimethylxanthine-RX.

Embodiment 16: The DPAs amine complex compound of any one of the above-mentioned embodiments, wherein the DPA-2-RX includes a 7-[2-[4-(2-methoxybenzene)piperazinyl]ethyl]-1,3-dimethylxanthine-RX.

Embodiment 17: The DPAs amine complex compound of any one of the above-mentioned embodiments, wherein the DPA-3-RX includes a 7-[2-[4-(4-nitrobenzene)piperazinyl]ethyl]-1,3-dimethylxanthine-RX.

Embodiment 18: The DPAs amine complex compound of any one of the above-mentioned embodiments, wherein the DPA-4-RX includes a 7-[2-[4-(2-nitrobenzene)piperazinyl]ethyl]-1,3-dimethylxanthine-RX.

### REFERENCE

Am J Physiol Endocrinol Metab. 2007 December; 293(6):E1736-45
Phytother Res. 2009 May; 23(5):713-8.
Prog Lipid Res. 2009 September; 48(5):275-97.
Am J Physiol Endocrinol Metab. 2010; 298(4):E846-53.
Drug Discovery Today: Disease Mechanisms 2010; 7(3-4): e175-e183
British Journal of Pharmacology.2011; 164,1248-126
Trends in Endocrinology and Metabolism. 2011; 22, 404-411

What is claimed is:

1. A method for inhibiting an obesity, comprising a step of:
administering to a subject in need an effective amount of a pharmaceutical composition comprising one of a DPAs derivative compound and a DPAs amine complex compound.

2. The method as claimed in claim 1, wherein the DPAs derivative compound is represented by 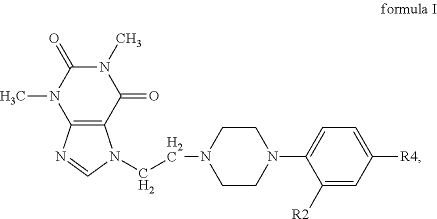
wherein R2 and R4 are each selected independently from the group consisting of a C1˜C5 alkoxy group, a hydrogen, a nitro group, and a halogen atom, and halogen atom is one selected from a group consisting of a fluorine, a chlorine, a bromine and an iodine.

3. The method as claimed in claim 1, wherein the DPAs derivative compound comprising one selected from a group consisting of a DPA-1, a DPA-2, a DPA-3 and a DPA-4.

4. The method as claimed in claim 3, wherein the DPA-1 includes a 7-[2-[4-(4-(2-chlorophenyl)piperazinyl]-ethyl]-1,3-dimethyl xanthine.

5. The method as claimed in claim 3, wherein the DPA-2 includes a 7-[2-[4-(4-(2-methoxybenzene)piperazinyl]-ethyl]-1,3-dimethylxanthine

6. The method as claimed in claim 3, wherein the DPA-3 includes a 7-[2-[4-(4-nitrobenzene)piperazinyl]-ethyl]-1,3-dimethylxanthine.

7. The method as claimed in claim 3, wherein the DPA-4 includes a 7-[2-[4-(2-nitrobenzene)piperazinyl]-ethyl]-1,3-dimethylxanthine.

8. The method as claimed in claim 1, wherein the DPAs amine complex compound is represented by 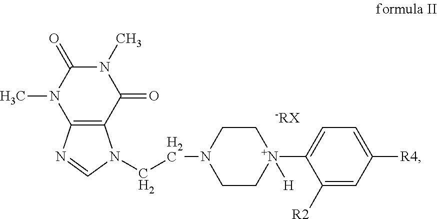
wherein each of R2 and R4 is one selected from a group consisting of a C1˜C5 alkoxy group, a hydrogen, a nitro group and a halogen atom, RX is a carboxylic group being one selected from a group consisting of a Statin derivative, a sodium carboxyl methylcellulose (sodium CMC), a poly-γ-polyglutamic acid (γ-PGA) derivative, a co-polymer and a combination thereof, and ⁻RX is an anion of the carboxylic group.

9. The method as claimed in claim 8, wherein the halogen atom is one selected from a group consisting of a fluorine, a chlorine, a bromine and an iodine.

10. The method as claimed in claim 8, wherein the poly-γ-polyglutamic acid (γ-PGA) derivative is one selected from a group consisting of an alginate sodium, a poly-γ-polyglutamic acid (γ-PGA), a poly-γ-polyglutamic acid sodium (γ-PGA sodium), a glutamic acid-L-lysine-L-tyrosine and a combination thereof.

11. The method as claimed in claim 8, wherein the co-polymer is one selected from a group consisting of a hyaluronic acid, a polyacrylic acid, a dextran sulfate, a polymethacrylates (PMMA), an Eudragit, a dextran sulfate, a heparan sulfate, a polylactic acid (PLA), a polylactic acid sodium (PLA sodium), a polyglycolic acid sodium (pga sodium) and a combination thereof.

12. The method as claimed in claim 8, wherein the Statin derivative is one selected from a group consisting of an Atorvastatin, a Cerivastatin, a Fluvastatin, a Lovastatin, a Mevastatin, a Pravastatin, an Rosuvastatin, a Simvastatin and a combination thereof.

13. The method as claimed in claim 1, wherein the obesity is associated with a disease or condition selected from the group consisting of Hyperlipidemia, feeding-rate of obesity, atherosclerosis, over weight of adipose tissues and unbalance metabolic homeostasis.

14. The method as claimed in claim 1, wherein the subject human and non-human animal.

15. A pharmaceutical composition comprising an effective amount of a DPAs amine complex compound represented by formula II: 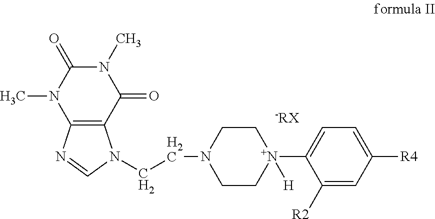
wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a co-polymer; and
⁻RX is an anion of a carboxylic group donated from one selected from a group consisting of a Statin, a sodium CMC, a poly-γ-polyglutamic acid (γ-PGA) derivative and a co-polymer.

16. The pharmaceutical composition as claimed in claim 15, wherein the halogen atom is one selected from a group consisting of a fluorine, a chlorine, a bromine and an iodine.

17. The pharmaceutical composition as claimed in claim 15, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

18. The pharmaceutical composition as claimed in claim 15, wherein the co-polymer includes one selected from a group consisting of a hyaluronic acid a polyacrylic acid, a dextran sulfate, a polymethacrylates (PMMA), an Eudragit, a dextran sulfate, a heparan sulfate, a polylactic acid (PLA), a polylactic acid sodium (PLA sodium) and a polyglycolic acid sodium (pga sodium).

19. The pharmaceutical composition as claimed in claim 15, wherein the Statin derivative includes one selected from a group consisting of an Atorvastatin, a Cerivastatin, a Fluvastatin, a Lovastatin, a Mevastatin, a Pravastatin, Rosuvastatin and a Simvastatin.
